(19) United States
(12) Patent Application Publication
(10) Pub. No.: US 2002/0042043 A1
(43) Pub. Date: Apr. 11, 2002

(54) Preparation of a pathogen inactivated solution of red blood cells having reduced immunogenicity

(76) Inventor: Adonis Stassinopoulos, Dublin, CA (US)

Correspondence Address: CERUS CORPORATION, 2525 Stanwell Drive # 300, Concord, CA, 94520, US

(21) Appl. No.: 09/872,466

(22) Filed: May 31, 2001

Related U.S. Application Data
(60) Provisional application No. 60/208,962, filed on May 31, 2000.

(51) Int. Cl. C12N 5/08

(52) U.S. Cl. 435/2; 435/372

(57) ABSTRACT

Compounds and methods are provided for the preparation of a red blood cell composition which has significantly reduced antigenicity and in which any possible pathogen contaminants have been substantially inactivated. The red blood cell compositions are of particular use for introduction into an individual in cases where the potential for an immune reaction is high, for example in alloimmunized blood recipients or in trauma situations where the possibility of transfusion of a mismatched unit of blood is higher. The red blood cell compositions of this invention provide a much lower risk of transfusion associated disease transmission as well as a much lower risk of a transfusion associated immune reaction.

### REFERENCE TO RELATED APPLICATIONS

This application claims priority to U.S. Provisional Patent Application Ser. No. 60/208,962, filed May 31, 2000; the disclosure of which is hereby incorporated by reference

### FIELD OF THE INVENTION

The present invention relates generally to compositions and methods for reducing risks associated with transfusing cellular compositions from one individual to another, and more particularly to reducing the risk of complications due to an immune response during such a transfusion as well as reducing the risk of transmitting disease during the transfusion. The present invention is primarily concerned with reducing the risk of an immune reaction and the risk of disease transmission in the transfusion of a red blood cell composition.

### BACKGROUND

Blood transfusions are essential in the treatment of patients with anemia, trauma, surgical bleeding and certain inherited disorders. Risks to a patient receiving a red blood cell transfusion include hemolytic transfusion reaction, febrile, non-hemolytic transfusion reaction, transfusion-related acute lung injury, alloimmunization, graft vs. host disease, and possible infection from viruses and bacteria that may be present in the blood product.

Hemolytic transfusion reactions may be due to reaction of a recipient antibody to an antigen on the donor red blood cells, often resulting from human error in transfusing a mismatched unit of red blood cells. Febrile, non-hemolytic transfusion reactions, which account for 90% of transfusion reactions, result from recipient antibodies reacting to an antigen on the donor leukocytes. Transfusion-associated lung injury is believed to be caused by leukocyte antibodies in the donor red blood cells resulting in the agglutination of recipient leukocytes.

Alloimmunization is of greatest risk to patients who require chronic red blood cell transfusions and results from the patient forming antibodies to minor group antigens on the red blood cells, which cause reactions in subsequent transfusions (allosensitization). Graft vs. host disease occurs when donor leukocytes engraft and proliferate in the recipient, and then react against tissue antigens of the host.

Elaborate systems of identification and testing are in place to ensure that correctly matched red blood cells are transfused. However, human performance is a factor in such systems and mistakes are made. A report on transfusion error in New York state indicates ABO incompatible transfusion of 1 in 33,000 units resulting in 3 fatalities [Linden et al., Transfusion 32:601 (1992)]. Similar error rates have been reported in Great Britain [McClelland et al., BMJ 308:1205 (1994)].

Possible experimental treatments to eliminate the need for ABO matching of blood include enzymatic removal of terminal sugar antigens which convert type A or B red blood cells to type O [Lenny et al., Transfusion 34:209 (1994), Lenny et al., Biotechnology of blood, Goldstein, J. ed. pp 75-100 (1991)]. Another experimental approach, potentially useful for infusion of red blood cells into alloimmunized patients, is to mask the red blood cell antigens by attaching long, flexible hydrophilic molecules such as polyethylene glycol to the surface of the red blood cells creating an essentially non-immunogenic red blood cell [Scott et al., Proc. Natl. Acad. Sci. USA 94:7566 (1997), PCT publication 99/16318]. The effects of the above mentioned processes on febrile, non-hemolytic transfusion reaction, transfusion-associated lung injury, and graft vs. host disease is unknown. However, the risk of these three reactions can be reduced by leukofiltration of the red blood cell product.

Transfusion of virus (e.g. HIV, hepatitis) or bacteria (e.g. Yersinia enterocolitica) contaminated blood is a major concern. While bacterial contamination happens rarely in red blood cells stored at 4° C., bacteria that like to grow in the cold (psychrophilic bacteria, e.g. Yersinia enterocolitica, Pseudomonas fluorescens, and Serratia marcescens) are the most common contaminants associated with bacterial sepsis after red blood cell transfusion [Gottlieb, Anaesth. Intens. Care 21:20 (1993)]. In order for bacteria to cause morbidity, a certain load needs to be present in the infused product since both a complement based and a phagocytosis based defense mechanism exists in the recipient's immune system. These mechanisms depend on the immunogenicity of the bacteria, the recipient's constitution, and the characteristics of the blood product (e.g. high or low plasma level, leukofiltration, etc.). Bacterial sepsis is due in part to the release of endotoxins from the bacteria. While the growth of psychrophilic bacteria is slowed at 4° C., storage of one to three weeks may result in sufficient build up of bacteria or endotoxins to cause severe transfusion reactions.

Pathogen inactivation of blood products is an important way of improving the safety of blood transfusions. While blood screening for viruses such as hepatitis B (HBV), hepatitis C (HCV) and human immunodeficiency virus (HIV) is routine, testing does not detect 100% of infected units and the possibility of infusing an infected unit due to human error is similar to the problem of ABO mismatched transfusions. In addition, unknown pathogens that are not tested for may be introduced into the blood supply, such as what occurred with HIV prior to its identification and testing. There is currently no testing for other pathogens such as bacteria or protozoans in blood. Parasites such as Trypanosoma cruzi, Babesia microti, and Leishmania donovani are transmitted by blood transfusion. Trypanosoma cruzi is endemic in Central and South America. Babesia microti is endemic to the United States and infections are particularly identified in regions around laboratories in which it is studied. Since there is no testing for these, the only protection against such protozoans in the United States is through donor screening.

The above-mentioned pegylation process for the masking of red blood cell antigens may be effective in the inactivation of certain pathogens. Inactivation of viral particles via polyethylene glycol modification is discussed in PCT publication 99/00145. It is not known what affect these antigen masking methods would have on other pathogens such as bacteria or protozoans. In particular, the effect of the antigen masking process on bacteria that may be present in red blood cells has not been addressed. It is likely that reactive groups on the surface of bacteria will also be modified by the polyethylene glycol resulting in a less immunogenic bacteria which could go unnoticed by a hosts immune system. Whether this bacteria is capable of dividing is unknown. If, in fact, such a modified bacteria were capable of growth, one can imagine that the progeny would also be less immunogenic and undetected by the host immune system until sufficient divisions have diluted out the effects of the polyethylene glycol modification. By this time, it is possible the bacteria will have reached a level that could not be controlled by the host immune system or their division has generated enough endotoxins to cause septic shock. In this scenario, a “stealth bacteria” has been created which may pose a greater risk than an unmodified bacteria. This suggests a need for pathogen inactivation in such a system beyond the need that already exists.

Several approaches to inactivation of pathogens in red blood cells exist. The use of phthalocyanines or thiazine dyes and visible light has been demonstrated [U.S. Pat. Nos. 5,232,844 and 5,827,644, the disclosure of which is hereby incorporated by reference]. The use of compounds that contain a nucleic acid binding ligand and a group that is reactive with a nucleic acid is described in PCT publications 96/39818 and 98/30545, corresponding to U.S. Pat. Nos. 171,177B1 and 6,093,725, respectively, the disclosures of which are hereby incorporated by reference. The use of polyanion selective ethyleneimine compounds is discussed in PCT publication 97/07674, and U.S. Pat. Nos. 6,136,586 and 6,093,564, the disclosures of which are hereby incorporated by reference. These methods have a potential disadvantage in that there is a possibility of forming neoantigens through side reactions with cellular membrane and plasma components. While this risk may be small, repeated use of such pathogen inactivated products may result in allosensitization.

The search for a blood substitute that has a reduced risk of disease transmission and is non-immunogenic is ongoing [Ketcham et al., Annals of Emergency Medicine 33:3 pp 326-337 (1999)]. While the above methods demonstrate various means of treating a red blood cell composition without adversely affecting the blood product, a singular method that produces a fully functioning red blood cell product which has a significantly reduced risk of being infectious and has significantly reduced immunogenicity would be beneficial, unique, and preferable to a blood substitute for most applications as it would have better oxygen transport properties and a longer in vivo survival time than most blood substitutes. Such a red blood cell product would be particularly suited for use in alloimmunized patients and in trauma situations where there is no time for crossmatching the blood.

### SUMMARY OF THE INVENTION

The present invention contemplates a composition comprising red blood cells that has a significantly reduced risk of being infectious, has significantly reduced immunogenicity, and is suitable for in vivo use. A preferred embodiment of the present invention relates to a composition comprising red blood cells suspected of containing a pathogen wherein the red blood cell composition has been treated such that the pathogen is substantially inactivated so as to significantly reduce the risk of being infectious and in which the red blood cell antigens are substantially masked so that the transfusion of the treated red blood cells into an antigen mismatched individual would result in a reduced immune reaction compared to the immune reaction of the tranfusion of an untreated red blood cell composition, where the treated red blood cell composition is suitable for in vivo use. In addition, the present invention relates to methods for the production of these red blood cell compositions in which a contaminating pathogen that may be present has been substantially inactivated such that the red blood cell compositions have a significantly reduced risk of being infectious and in which the red blood cell antigens are substantially masked so as to suitably reduce the risk of eliciting any transfusion reaction associated with an immune response to such antigens.

The present invention further contemplates methods of producing the red blood cell compositions discussed above. In a preferred embodiment, the method comprises the steps of 1) treating a cell composition in order to substantially inactivate a pathogen that may be present in the red blood cell composition and 2) subsequently treating the red blood cell composition in order to substantially mask antigens so as to significantly reduce the immunogenicity of the red blood cells in the composition such that the resulting treated red blood cell composition remains functional for it's intended use, such as for transfusion. In another embodiment, the process can be carried out from step 1 to step 2 without additional manipulation of the sample, i.e. without dilution, washing, addition of buffers, etc. In another embodiment, the treated red blood cell composition is additionally washed or treated to reduce the level of any unwanted reagents of the process or side products of these reagents, or unwanted by-products of the process.

In another embodiment, the method comprises the steps of 1) treating a red blood cell composition in order to substantially mask antigens so as to significantly reduce the immunogenicity of the red blood cells in the composition and 2) subsequently treating the red blood cell composition in order to substantially inactivate a pathogen that may be present in the red blood cell composition such that the resulting treated red blood cell composition remains functional for it's intended use, such as for transfusion. In another embodiment, the process can be carried out from step 1 to step 2 without additional manipulation of the sample, i.e. without dilution, washing, addition of buffers, etc. In another embodiment, the treated red blood cell composition is additionally washed or treated to reduce the level of any unwanted reagents of the process or side products of these reagents, or unwanted by-products of the process.

In another embodiment, the method comprises concurrently treating a red blood cell composition in order to substantially mask antigens so as to significantly reduce the immunogenicity of the red blood cells in the composition and treating the red blood cell composition in order to substantially inactivate a pathogen that may be present in the red blood cell composition such that the treated red blood cell composition remains functional for it's intended use, such as for transfusion. In another embodiment, the treated red blood cell composition is additionally washed or treated to reduce the level of any unwanted reagents of the process or side products of these reagents, or unwanted by-products of the process.

In another embodiment, the present invention contemplates an ex vivo method of treating a red blood cell composition suspected of containing a pathogen comprising, in any order, contacting the red blood cell composition with a compound that can inactivate the pathogen, under conditions that result in substantial inactivation of the pathogen, and contacting the red blood cell composition with a compound that can bind to the red blood cells and substantially mask red blood cell antigens so as to significantly reduce the immunogenicity of the red blood cells, under conditions that result in a red blood cell composition that, if transfused into an antigen mismatched animal, would result in a reduced immune reaction as compared to the immune reaction of an untreated red blood cell composition, wherein the resulting red blood cell composition is suitable for in vivo use. In another embodiment, the treated red blood cell composition is additionally washed or treated to reduce the level of any unwanted reagents of the process or side products of these reagents, or unwanted by-products of the process.

### DESCRIPTION OF THE DRAWINGS

FIG. 1 is an exemplary plot of the population maximum fluorescense by flow cytometry for MPEG modified red blood cells.

### DESCRIPTION OF THE INVENTION

The present invention generally relates to new compositions comprising red blood cells which have a significantly reduced risk of being infectious, have significantly reduced immunogenicity, and are suitable for in vivo use, e.g. for transfusion. The present invention also relates to in vitro or ex vivo methods of treating a composition comprising red blood cells, the methods resulting in a composition comprising the treated red blood cells which has a significantly reduced risk of being infectious, has significantly reduced immunogenicity, remains functional, and is suitable for in vivo use. In accordance with the present invention, a first compound having a nucleic acid binding ligand and a group that is reactive with the nucleic acid is selectively employed to treat possible contamination of a red blood cell composition by nucleic acid containing organisms, including pathogenic viruses, bacteria, and parasites, while a second compound comprising a non-immunogenic group having a suitable reactive coupling group is selectively employed to mask red blood cell antigens by covalently binding to the surface of the red blood cells resulting in a red blood cell composition having significantly reduced immunogenicity. In some embodiments a quencher is included which would effectively react with any excess compounds of the present invention, both the pathogen inactivating compound and the reactive antigen masking compound. Additionally, a process of removing any excess compounds, products of the compound's reaction with a quencher, side products of the process, and the quencher itself are contemplated in the present invention.

A nucleic acid containing pathogen is defined as any agent capable of causing disease in a human, other mammals, or vertebrates that contains nucleic acid as genetic material. Examples include microorganisms such as unicellular or multicellular microorganisms. Examples of pathogens are bacteria, viruses, protozoa, fungi, yeasts, molds, and mycoplasmas that cause disease in humans, other mammals, or vertebrates. The genetic material of the pathogen may be DNA and/or RNA, and the genetic material may be present as single-stranded or double-stranded nucleic acid or mixtures thereof. The nucleic acid of the pathogen may be in solution, intracellular, extracellular, or bound to cells.

Pathogen inactivation is defined as rendering pathogens in a material incapable of reproducing. Inactivation is expressed as the negative logarithm of the fraction of remaining pathogens capable of reproducing. For example, if a compound at a certain concentration renders 90% of the pathogens in a material incapable of reproduction, 10% or one-tenth (0.1) of the pathogens remain capable of reproduction. The negative logarithm of 0.1 is 1, and that concentration of that compound is said to have inactivated the pathogens present by 1 log, or the compound is said to have 1 log kill at that concentration. The log inactivation can also be viewed as the comparison of pathogen titer in a control sample to a treated sample, where the log of the ratio of control titer to titer remaining after inactivation represents the log inactivation. For example, if a control titer measures 10⁷ (i.e. a 10⁷ dilution of the solution results in no detection of the pathogen where a 10⁶ dilution results in detection) and a treated sample titer measures 10²(i.e. a 10² dilution of the solution results in no detection of the pathogen where a 10¹ dilution results in detection), the resulting level of inactivation is 5 logs.

In vivo use of a material or compound is defined as introduction of the material or compound into a living individual. For example, the transfusion of a blood product into an individual in need of a transfusion would be considered an in vivo use of the blood product. An individual, as defined herein, is a vertebrate, preferably a mammal, including domestic animals, sport animals, and primates, including humans.

In vitro use of a material or compound is defined as a use of the material or compound outside a living individual, where typically neither the material nor compound is intended for reintroduction into a living individual. Ex vivo use of a compound is defined as using a compound for treatment of a biological material outside a living individual, where the treated biological material is intended for use inside a living individual. For example, removal of blood from a human and introduction of a compound into that blood to inactivate pathogens is defined as an ex vivo use of that compound if the blood is intended for reintroduction into that human or another human. Reintroduction of the human blood into that human or another human would be in vivo use of the blood, as opposed to the ex vivo use of the compound. If the compound is still present in the blood when it is reintroduced into the human, then the compound, in addition to its ex vivo use, is also introduced in vivo.

The compositions considered in the present invention are considered to be functional if certain in vitro and in vivo properties are similar to the properties of a sample that is not treated by the methods of the present invention. In addition, there are certain blood banking standards that must be met by compositions of the present invention. To be suitable for in vivo use, the compositions must also exhibit low levels of toxicity, as measured for example by gene toxicity, animal studies and Ames mutagenicity assays.

The present invention contemplates the antigen masking or immune masking of red blood cells. Red blood cells comprise several antigenic determinants on their surface that might cause an immune response. For example, the immune system of a recipient of a red blood cell transfusion may recognize certain antigens on the transfused red blood cell as foreign and mount an immune response to the red blood cells. Masking of these antigens involves the modification or hiding of these antigens so that they are no longer accessible to or recognized by the immune system of the recipient. Certain compounds may be linked to the red blood cell surface such that the antigens on the red blood cell surface are hidden or masked by the compound. In addition, these compounds that can be linked to the red blood cell surface may have a structure that is not itself recognized by an immune system, i.e. these compounds are non-immunogenic or non-antigenic. By masking antigens in this manner, the transfused red blood cells are no longer capable of eliciting an immune response from the recipient. Red blood cell antigens are considered to be substantially masked when the treated red blood cells have significantly reduced reactivity toward antibodies that bind specific red blood cell antigens when compared to the binding of an untreated red blood cell. This reduced immunogenicity can be readily measured using in vitro antibody binding assays or in vitro measurements of the amount of modification of the red blood cells.

It is further contemplated that the red blood cells are suspected of containing a pathogen, such as bacteria or parasite. In the process of masking the red blood cell antigens it is possible to also mask antigens on such bacteria or parasite. These antigens are important in the recognition and elimination of such pathogens by an individual's immune system. Under these conditions, a red blood cell composition which has been treated to mask red blood cell antigens and is transfused into an individual may result in contamination of that individual by bacteria or parasite that is not recognized by the individual's immune system. In the case of bacteria, an individual might not have an immune response to the bacteria such that the bacteria may produce endotoxin or may subsequently divide within the individual's bloodstream to levels that pose a significant health risk to that individual, wherein the risk of infection from the bacteria is higher than if the red blood cell composition had not been treated to mask the antigens. In the case of parasites, depending on what stage of the life cycle the parasite is in, the masking of antigens might hide the parasite from an individual's immune system resulting in a greater health risk from the parasite than from a parasite that has not been treated to mask the antigens.

Quenchers as used herein refer to compounds that are capable of reacting with pathogen inactivating compounds and/or reactive immune masking compounds encompassed by the present invention. The pathogen inactivation compounds of the present invention target nucleic acid of the pathogen. While this reactivity is favored, other unwanted reactions may occur as well. The purpose of a quencher is to provide another pathway for the pathogen inactivation compounds which is preferred over the unwanted side reactions such that the majority of the pathogen inactivation compound reacts with either the intended nucleic acid target or with the quencher. In another embodiment, the quencher may serve to react with any excess material, whether it is pathogen inactivation compound or reactive immune masking compound, after the compound has sufficiently reacted with its intended target.

Compound reduction devices as used herein are devices which are intended to remove unwanted compounds, for example unreacted pathogen inactivation or antigen masking compounds or by-products of the pathogen inactivation and antigen masking process, such as the products of reaction of the compounds with quenchers or endogenous materials. Such reduction devices comprise an adsorbent material in a suitable matrix, wherein the adsorbent is selected to preferentially bind the unwanted compounds without binding essential components that may be necessary for the red blood cells to function properly.

Alkyl as used herein refers to a cyclic, branched, or straight chain chemical group containing carbon and hydrogen, for example methyl, pentyl, and adamantyl. Alkyl groups can either be unsubstituted or substituted with one or more substituents, e.g., halogen, alkoxy, acyloxy, amino, hydroxyl, thiol, carboxy, benzyloxy, phenyl, benzyl, or other functionality. Alkyl groups can be saturated or unsaturated (e.g., containing —C═C— or —C≡C— subunits), at one or several positions. Usually, alkyl groups will comprise 1 to 12 carbon atoms, preferably 1 to 10 carbon atoms, and more preferably 1 to 8 carbon atoms, unless otherwise specified.

Heteroalkyl as used herein are alkyl chains with one or more heteroatoms incorporated into the chain. Heteroatoms include N, O, S, and P. Heteroatoms also include oxidized forms of the heteroatoms N, S and P. Examples of heteroalkyl groups include (but are not limited to) methoxy, ethoxy, and other alkyloxy groups; ether containing groups; amide containing groups such as polypeptide chains; ring systems such as piperidinyl, lactam and lactone; and other groups which incorporate heteroatoms into the carbon chain. Typically, heteroalkyl groups will comprise, in addition to the heteroatom(s), 1 to 12 carbon atoms, preferably 1 to 10 carbon atoms, and more preferably 1 to 8 carbon atoms, unless otherwise specified.

Aryl or Ar refers to an unsaturated aromatic carbocyclic group having a single ring (e.g., phenyl) or multiple condensed rings (e.g., naphthyl or anthryl), which can be optionally unsubstituted or substituted with amino, hydroxyl, alkyl (e.g. C₁₋₈ alkyl), alkoxy, halo, thiol, and other substituents.

Heteroaryl groups are unsaturated aromatic carbocyclic groups having a single ring (e.g., pyridyl or furyl) or multiple condensed rings (e.g., acridinyl, indolyl or benzothienyl) and having at least one hetero atom, such as N, O, or S, within at least one of the rings. The ring(s) can optionally be unsubstituted or substituted with amino, hydroxyl, alkyl, alkoxy, halo, thiol, acyloxy, carboxy, benzyloxy, phenyl, benzyl, and other substituents.

I. Compounds for Inactivating Pathogens in a Red Blood Cell Composition

The present invention contemplates treating a composition comprising red blood cells ranging in hematocrit from about 1% to about 65% or higher with a compound that will inactivate a pathogen that may be present in the composition. The compounds used ideally will inactivate a pathogen in the red blood cells over a period of several minutes to hours depending on the compound. Compounds contemplated for use in the present invention include those known and used in the art for the inactivation of pathogens in red blood cells, such as phthalocyanine or thiazine dyes (for example methyelene blue and related dyes), riboflavin and related compounds, and ethyleneimine oligomers and related compounds. Preferred compounds will react preferentially with nucleic acids relative to other elements present in a red blood cell composition, such as proteins and cellular membranes. It is considered that some compounds of the present invention may need additional activation energy to react with the nucleic acid, for example, by irradiation with a suitable wavelength source (e.g. photoactivated compounds such as methylene blue and riboflavin). Inactivation of pathogens is defined as rendering pathogens in a material incapable of reproducing. It is not essential that total inactivation (i.e. all pathogens present are incapable of reproducing) occurs, only that substantially all of the pathogen present is inactivated so that the amount of pathogen remaining is insufficient to cause disease in a normal individual. A normal individual is one who is not pathologically immunosuppressed as the result of a disease or immuno suppressive treatment. The pathogen inactivation treatment preferably results in 1 log of inactivation of the pathogen, more preferably 2 logs of inactivation, even more preferably 3 logs of inactivation, and in many circumstances 4 logs or higher, including 6 logs of inactivation of pathogen.

Without intending to be limited to any particular mechanism of action of the present invention, compounds for inactivation of pathogens preferentially react with nucleic acids relative to other elements that may be present. In general, preferred compounds have two common characteristics. First, they have an affinity for nucleic acids, e.g. by non-covalent binding to nucleic acids. Second, they react with the nucleic acid so as to render it incapable of replication. In one embodiment, the compounds for inactivation have a moiety that has an affinity for nucleic acids that is not distinguishable from the moiety that reacts with the nucleic acid. In another embodiment, the compounds may have a moiety that has an affinity for nucleic acids and a moiety that is reactive with the nucleic acid. Such compounds have an anchor portion that has an affinity for the nucleic acid (i.e. it has the ability to bind nucleic acid non-covalently) linked to an effector portion which has the ability to form a covalent bond to the nucleic acid (i.e. the effector portion reacts to bind the nucleic acid covalently). In another embodiment of the compounds for inactivation, a non-covalent nucleic acid binding anchor portion is linked to the nucleic acid reactive effector portion by a linker which can be hydrolyzed so as to no longer link the anchor portion with the effector portion. This latter type of linker is termed “frangible”.

### Compounds with Anchor-effector Groups

1. Non-covalent Nucleic Acid Binding Anchor Group

A compound which binds nucleic acid has a “nucleic acid binding ligand”, herein defined as a group that has an affinity for nucleic acids and can bind to nucleic acids non-covalently. There are several modes of binding to nucleic acids. Compounds that bind by any of the following modes, combinations of them, or other modes are considered nucleic acid binding ligands and can be used as an anchor portion. While the invention is not limited to the following compounds, some examples of nucleic acid binding ligands are: a) intercalators, such as acridines, acridones, proflavine, acriflavine, actinomycins, anthracyclinones, rhodomycins, daunamycin, thiaxanthenones, miracil D, anthramycin, mitomycins, echinomycin, quinomycin, triostin, diacridines, ellipticene (including dimers, trimers and analogs), norphilin A, fluorenes and fluorenones, fluorenodiamines, quinacrines, benzacridines, phenazines, phenanthradines, phenothiazines, chlorpromazine, phenoxazines, benzothiazoles, xanthenes and thioxanthenes, anthraquinones, anthrapyrazoles, benzothiopyranoindoles, 3,4-benzopyrene, benzopyrene diol epoxide, 1-pyrenyloxirane, benzanthracenes, benzodipyrones, benzothiazoles, quinolines (e.g. chloroquine, quinine, phenylquinoline carboxamides), furocoumarins (e.g. psoralens and isopsoralens), ethidium, propidium, coralyne, ellipticine cation and derivatives, polycyclic aromatic hydrocarbons and their oxirane derivatives; b) minor groove binders such as distamycin, netropsin, other lexitropsins, Hoechst 33258 and other Hoechst dyes, DAPI (4′,6′-diamidine-2-phenylindole), berenil, and triarylmethane dyes; c) major groove binders such as aflatoxins; d) molecules that bind by electrostatics (phosphate backbone binders), such as spermine, spermidine, and other polyamines; e) nucleic acids or analogues which bind by such sequence specific interactions as triple helix formation, D-loop formation, and direct base pairing to single stranded targets. Derivatives of these compounds are also non-limiting examples of nucleic acid binding ligands, where a derivative of a compound includes, but is not limited to, a compound which bears one or more substituents of any type at any location, oxidation or reduction products of the compound, etc.

2. Nucleic Acid Reactive Effector Group

A nucleic acid reactive effector group is a group that reacts to cause a covalent modification of a nucleic acid. A preferred embodiment of a nucleic acid reactive effector group is a mustard group, herein defined as including mono or bis-(haloethyl)amine groups, and mono haloethylsulfide groups. A further embodiment of possible effector groups includes mustard equivalents, defined as groups that react by a mechanism similar to the mustards (i.e. by forming reactive intermediates such as aziridinium or aziridine complexes and sulfur analogs of these complexes). Examples of such mustard equivalents includes aziridine derivatives, mono or bis-(mesylethyl)amine groups, mono mesylethylsulfide groups, mono or bis tosylethylamine groups, and mono tosylethylsulfide groups. The present invention is not limited strictly to mustards and mustard equivalents. Additional examples of effectors include, but are not limited to, epoxides, aldehydes, aldehyde synthons, and other alkylating and cross-linking agents. Aldehyde synthons are defined as any compound that breaks down to formaldehyde in aqueous solution. While the compounds for inactivation of pathogens are not limited to any specific mechanism, the effector groups, which are, or are capable of forming an electrophilic group, such as a mustard group, are believed to react with and form a covalent bond to nucleic acid.

An embodiment of a compound containing a nucleic acid binding anchor group and an alkylating effector group is quinacrine mustard (Aldrich Chemical), the structure of which is shown below.

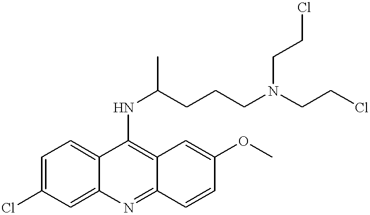

### B. Anchor-Effector with Frangible Linking Group.

Examples of linkers that can be used to join the anchor and effector groups with a labile bond (referred to as frangible linkers) include, but are not limited to, compounds having functional groups such as an ester or thioester (where the carbonyl carbon of the ester or thioester may be between the anchor and the sp³ oxygen or sulfur of the ester or thioester, respectively (forward ester) or where the sp³ oxygen or sulfur of the ester or thioester is between the anchor and the carbonyl carbon of the ester or thioester, respectively (reverse ester), forward and reverse thionoester, forward and reverse dithioic acid, sulfate, forward and reverse sulfonates, phosphate, and forward and reverse phosphonate groups. Thioester designates the —C(═O)—S— group, thionoester designates the —C(═S)—O— group and dithioic acid designates the —C(S═)—S— group. The frangible linker may also include an amide, where the carbonyl carbon of the amide is between the anchor and the nitrogen of the amide (forward amide), or where the nitrogen of the amide is between the anchor and the carbonyl carbon of the amide (reverse amide). The functional linker groups that are designated forward are oriented such that, after hydrolysis, the resulting acidic function is covalently linked to the anchor portion and the resulting alcohol, amine or thiol function is covalently linked to the effector portion. The functional linker groups that are designated reverse are oriented such that, after hydrolysis, the resulting acidic function is covalently linked to the effector portion and the resulting alcohol, amine or thiol function is covalently linked to the anchor portion. The frangible linker may also be capable of degrading under conditions of enzymatic degradation, by endogenous enzymes in the red blood cell composition, or by enzymes added to the material.

A preferred embodiment which additionally contains a frangible linking group is exemplified by the structures shown below.

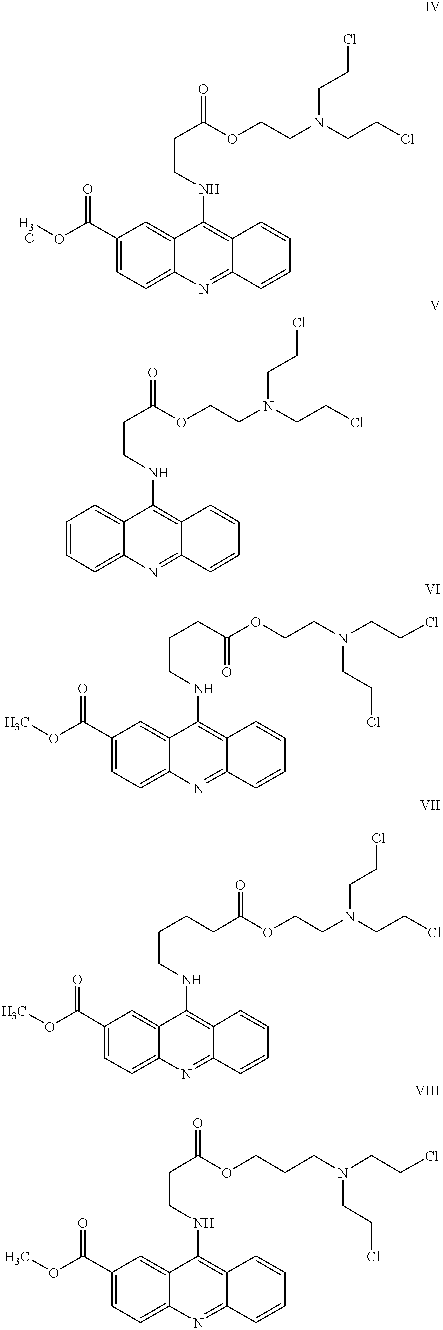

Additional embodiments of the compounds for inactivation of pathogens containing a frangible linker are described in PCT publication 98/30545, which corresponds to U.S. Pat. No. 6,093,725.

II. Compounds for Masking Immunogenicity of the Red Blood Cells

The present invention further contemplates treating a composition comprising red blood cells ranging in hematocrit from about 1% to about 65% or higher with a compound that will covalently bind to the surface of the red blood cells and substantially mask any antigens present on the cells. The conditions for reacting such antigen masking compounds with a composition comprising red blood cells may be compatible with the conditions of pathogen inactivation. In another embodiment, the conditions for reacting such compounds with a composition comprising red blood cells may be compatible, upon further adjustment of reaction conditions prior to or following the covalent binding to the red blood cells, with the conditions of pathogen inactivation. In a preferred embodiment, it is contemplated that prior to treating with an antigen masking compound, the red blood cell composition may be washed to reduce the level of plasma proteins in order to decrease competitive processes that may affect the level of antigen masking of the red blood cells. In addition to the removal of proteins, washing of the red blood cells with a buffer of the appropriate pH eliminates (quenches) the buffering capacity of the hemoglobin inside the red blood cells. The washing of the red blood cells prevents the gradual alteration of the pH from the optimal higher than physiologic value of approximately 8-9 to the physiological value of approximately 7. This lowering or buffering of the pH results in the reduction of the reaction rate of the activated antigen masking compound with the red blood cell surface and the reduction of the amount of modification achieved before the activated antigen masking compound decomposes.

Without intending to be limited to any particular mechanism of action of the present invention, compounds for covalent binding to red blood cells in order to mask antigens will comprise a non-immunogenic group and a coupling group. In one embodiment, upon linking the non-immunogenic group to a target molecule, a portion of the coupling group remains. In another embodiment, the coupling group is such that the non-immunogenic group is directly linked to a target molecule, leaving no portion of the coupling group.

In one embodiment, the non-immunogenic portion of the compound is polyethylene glycol (PEG) or a derivative of polyethylene glycol. A thorough discussion of the use of polyethylene glycol technology can be found in PCT publication 95/06058, hereby incorporated by reference. Additional compounds suitable for the present invention would comprise other non-immunogenic groups capable of blocking recognition of antigens on a red blood cell surface when covalently attached to the red blood cell surface. Such non-immunogenic groups include, but are not limited to, polyalkylene glycols (such as PEG, polypropylene glycol, mixed polypropylene-polyethylene glycols), polyalkylene glycol derivatives (such as methoxypolyethylene glycol, MPEG), polysaccharides (such as dextrans, cellulosics, Ficoll, and arabinogalactan), and hydrophilic, synthetic polymers such as polyurethanes.

Preferred compounds comprise polyethylene glycol and derivatives of polyethylene glycol attached to a suitable coupling group. Such polyethylene glycol compounds are also referred to as activated polyethylene glycol compounds and have the general formula Cp—(OCH₂CH₂)ₙ—OH wherein n is greater than or equal to 3 and Cp represents a coupling group which reacts with terminal thiol or amine groups on a red blood cell surface to covalently link the non-immunogenic group to the red blood cell. The molecular weight can vary up to approximately 200,000 or more. Preferred derivatives have a molecular weight range of 150 to 10,000, more preferably 500 to 10,000, most preferably 2,000 to 8,000. Derivatives wherein the end groups are modified include, but are not limited to, PEG ethers (e.g.Cp—(OCH₂CH₂)ₙ—OR, such as Cp—(OHC₂CH₂)ₙ—OCH₃ (MPEG)), PEG esters (e.g. Cp—(OCH₂CH₂)ₙ—OOCR, such as Cp—OCH₂CH₂)ₙ—OOC(CH₂)₁₄ CH₃), PEG amides (e.g. Cp—(OCH₂CH₂)ₙ—OOC(CH₂)₇CONHR), PEG amines (e.g. Cp—(OCH₂CH₂)ₙ—NH₂,), PEG acids (e.g. Cp—(OCH₂CH₂)ₙ—OCH₂COOH), PEG aldehydes (e.g. H—(OCH₂CH₂)ₙ—OCH₂CHO), and electrophilic derivatives such as halogenated PEG (e.g. H—(OCH₂CH₂)ₙ—Br. The preferred derivatives of the present invention are those of MPEG. Another embodiment of the present invention contemplates branched PEG and branched PEG derivatives in which PEG arms are linked giving multi armed branched molecules. Another embodiment of the present invention contemplates a mixture of two or more of the above mentioned types of compounds. A further embodiment contemplates a mixture of two or more of these types of activated PEG and PEG derivative compounds in which the coupling group is targetd to different nucleophiles on the red blood cell surface. For example, a mixture of activated MPEGs may be used wherein one MPEG may be activated with a coupling group that preferably reacts with an amine group while another MPEG may be activated with a coupling group that preferably reacts with a thiol group.

In one embodiment, the coupling group for linking the non-immunogenic group to the red blood cells comprises a reactive group which reacts with terminal thiol or amine groups on the red blood cell surface. Examples include, but are not limited to, sulphonate esters, substituted triazines, N-hydroxysuccinimide esters, anhydrides, substituted phenyl carbonates, oxycarbonylimidazoles, maleimides, aldehydes, glyoxals, carboxylates, vinyl sulphones, epoxides, mustard, mustard equivalents, isocyanates, disulphides, acrylates, allyl ethers, silanes, and cyanate esters. Mustards are herein defined as including mono or bis-(haloethyl)amine groups, and mono haloethylsulfide groups. Mustard equivalents are herein defined as groups that react by a mechanism similar to the mustards (i.e. by forming reactive intermediates such as aziridinium or aziridine complexes and sulfur analogs of these complexes). Examples of such mustard equivalents includes aziridine derivatives, mono or bis-(mesylethyl)amine groups, mono mesylethylsulfide groups, mono or bis tosylethylamine groups, and mono tosylethylsulfide groups. Other possible coupling groups are selected from 2,2,2-trifluoroethanesulphonate, pentafluorobenzenesulphonate, fluorosulphonate, 2,4,5-trifluorobenzenesulphonate, 2,4-difluorobenzenesulphonate, 2-chloro-4-fluorobenzenesulphonate, 3-chloro-4-fluorobenzenesulphonate, 4-amino-3-chlorobenzenesulphonate, 4-amino-3-fluorobenzenesulphonate, o-trifluoromethylbenzenesulphonate, m-trifluoromethylbenzenesulphonate, 2-trifluoromethoxybenzenesulphonate, 4-trifluoromethoxybenzenesulphonate, 5-fluoro-2-methylbenzenesulphonate, 4,6-dichlorotriazine, 6-chlorotriazine, N-hydroxysuccinimidyl succinate, N-hydroxysuccinimidyl glutarate, N-hydroxysuccinimidyl succinamide, N-hydroxysuccinimidylalkanedioicamides, N-hydroxysuccinimidyl derivatives of carboxymethylated polymers, N-hydroxysuccinimidyl esters of amino acids, succinimidylcarbonate, succinate mixed anhydride, succinic anhydride, 2,4,5-trichlorophenol, trichlorophenyl carbonate, nitrophenyl carbonate, 4-nitrophenol, cyanuric chloride, maleimide, N-substituted maleimide, acetaldehyde, propionaldehyde and chemically equivalent sulfur analogs, glyoxal, phenylglyoxal, acrylate, and methacrylate. In another embodiment, the coupling group is a halogen atom, preferably iodide, bromide or chloride.

A preferred embodiment of an activated non-immunogenic compound is 2,2,2-trifluoroethanesulphonylmonomethoxy polyethylene glycol (Tresyl MPEG, or TMPEG). Other preferred embodiments of an activated non-immunogenic compound are N-hydroxy succinimide propionic acid (SPA) and N-hydroxy succinimide butanoic acid (SBA) derivatives of MPEG, the structures of which are given below, where n is greater than or equal to 1, preferably greater than or equal to 3.

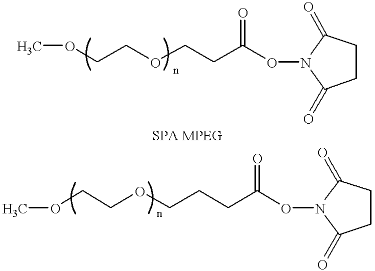

SBA MPEG

III. Compounds for Quenching of Unwanted Side Reactions

The present invention further contemplates the use of a quencher, which is intended to reduce unwanted side reactions of both the pathogen inactivation compounds and immune masking compounds. In a further embodiment, one, or a combination of quencher compounds may be used. The quencher or combination of quenchers may be added to the red blood cell composition prior to, simultaneously with, or after the addition of either the pathogen inactivating compound or the immune masking compound. In another embodiment, a particular quencher may be used to react with the pathogen inactivation compound while another quencher is used to react with the immune masking compound. In this instance, the quencher for the inactivation compound may be added prior to, simultaneously with, or after the addition of the inactivation compound while the quencher for the immune masking compound may be added prior to, simultaneously with, or after the addition of the immune masking compound. In another embodiment, if the inactivation compound is added to the red blood cell composition and quenched prior to addition of the immune masking compound, the amount of quencher may be reduced prior to addition of the immune masking compound. The reduction of quencher may be by washing the red blood cells or by treating with a device that selectively removes unwanted materials such as the quencher and unreacted or side products of the inactivation process. Similarly, if the immune masking compound is added to the red blood cell composition and quenched prior to addition of inactivation compound, the amount of quencher may be reduced prior to addition of the inactivation compound. The reduction of quencher may be by washing the red blood cells or by treating with a device that selectively removes unwanted materials such as the quencher and unreacted or side products of the immune masking process. Removal of quencher after one process but before the other may be done even if the quencher for each process is the same compound.

Preferred quenchers would include a nucleophilic group capable of reacting with the electrophilic groups of the pathogen inactivation compounds or the immune masking compounds. Examples of nucleophilic groups include, but are not limited to, thiol, thioacid, dithioic acid, thiocarbamate, dithiocarbamate, amine, phosphate, and thiophosphate groups. Additionally, the nucleophilic group could be an amino group, polyamino group, or a combination of thio and amino groups which could quench both unreacted pathogen inactivation compound and unreacted antigen masking compound. The quencher may be, or contain, a nitrogen heterocycle such as pyridine. The quencher can be a phosphate containing compound such as glucose-6-phosphate. The quencher also can be a thiol containing compound, including, but not limited to, glutathione, cysteine, N-acetylcysteine, mercaptoethanol, dimercaprol, mercaptan, mercaptoethanesulfonic acid and salts thereof (e.g. MESNA), homocysteine, aminoethane thiol, dimethylaminoethane thiol, dithiothreitol, and other thiol containing compounds. The quenchers also can be in the form of a salt, such as sodium or hydrochloride salt.

Other thiol containing compounds include, but are not limited to, methyl thioglycolate, thiolactic acid, thiophenol, 2-mercaptopyridine, 3-mercapto-2-butanol, 2-mercaptobenzothiazole, thiosalicylic acid and thioctic acid. Exemplary aromatic thiol compounds include 2-mercaptobenzimidazolesulfonic acid, 2-mercapto-nicotinic acid, napthalenethiol, quinoline thiol, 4-nitro-thiophenol, and thiophenol. Other quenchers include, but are not limited to, nitrobenzylpyridine and inorganic nucleophiles such as selenide salts or organoselenides such as selenocysteine, thiosulfate, sulfite, sulfide, thiophosphate, pyrophosphate, hydrosulfide, and dithionite. The quencher also can be a peptide compound containing a nucleophilic group. For example, the quencher may be a cysteine containing compound, for example, a dipeptide, such as GlyCys, or a tripeptide, such as glutathione or an amine containing compound such as polylysine. It is possible that the quencher may contain different nucleophilic groups, each of which are capable of quenching, such as the amine and thiol groups of glutathione.

IV. Devices for the Reduction of Unwanted Compounds From the Red Blood Cell Composition.

The present invention further contemplates devices and methods for the reduction of unwanted compounds from the red blood cell composition. These compounds include unreacted inactivation and antigen masking compounds, unwanted side products of these compounds, side products resulting from the reaction of these compounds with quencher, excess quencher and quencher side products, and unwanted by-products of the processes. Removal devices contemplated for use in the present invention comprise an adsorbent material in a suitable matrix, which specifically and selectively reduces the concentration of unwanted compounds without significant effects on the in vitro or in vivo properties of the red blood cell composition. A thorough discussion of devices and methods for the reduction of compounds that could be applied to some of the compounds of the present invention can be found in PCT publication WO 98/30327, hereby incorporated by reference. While it is an embodiment that such compound reduction devices be incorporated after all reactions are complete, the present invention is not limited as to when the reduction step or steps are incorporated into the process. The present invention is also not limited as to the number of reduction steps or the number of compound reduction devices to be used for these steps. In one embodiment of the present invention, the composition of the compound reduction device for inactivation compounds is different from the composition of the compound reduction device for the antigen masking compounds. It is contemplated that a compound reduction step be incorporated after addition of the inactivation compound but before addition of the antigen masking compound, with an optional reduction step following the reaction of the antigen masking compound. Similarly, a compound reduction step may be incorporated after addition of the antigen masking compound but before the addition of the inactivation compound, with an optional reduction step following the reaction of the inactivation compound.

V. Red Blood Cell Compositions Having Reduced Risk of Being Infectious and Reduced Immunogenicity and Methods of Their Preparation.

The compositions contemplated by the present invention include a red blood cell composition which has been treated to substantially inactivate a pathogen that may be present and to provide a red blood cell which has been treated to substantially mask antigens so as to have significantly reduced immunogenicity when transfused to a recipient. In another embodiment, the composition of red blood cells comprises red blood cells suspected of containing a pathogen wherein the red blood cell composition has been treated with a compound having an affinity for nucleic acids and an effector group that reacts to bond covalently to the nucleic acid such that the pathogen is substantially inactivated and wherein the red blood cell composition has been reacted with an antigen masking compound such that the red blood cell antigens are substantially masked such that the transfusion of the treated red blood cells into an antigen mismatched animal would result in a reduced immune reaction compared to the immune reaction of the transfusion of an untreated red blood cell composition, wherein the treated red blood cell composition is suitable for in vivo use. A further embodiment contemplates that the function of the red blood cell composition is not significantly reduced from that of a comparable untreated red blood cell composition. In particular, the composition is suitable for in vivo use in that the in vivo function is not lowered significantly relative to an untreated composition. An additional embodiment of the present invention is a medicament comprising red blood cells suspected of containing a pathogen wherein the red blood cells have been treated such that the pathogen is substantially inactivated and wherein red blood cell antigens are substantially masked so that the transfusion of the treated red blood cells into an antigen mismatched animal would result in a reduced immune reaction compared to the immune reaction of transfusion of an untreated red blood cell composition.

Parameters for suitability are known to those of skill in the art and include, but are not limited to, in vivo survival and certain in vitro parameters useful in assessing red blood cell function. For example, it is desirable that the function of the treated red blood cell composition is such that the in vivo survival of the red blood cells after circulating 24 hours post transfusion is greater than approximately 40%, more preferably 50%, and more preferably 75%, in other embodiments, this survival rate of approximately 40%, more preferably 50%, and more preferably 70%is maintained 24 hours post transfusion of the treated red blood cells, which have been stored prior to transfusion for up to 7 days, 14 days, 21 days, 35 days, and 42 days at 4° C. In addition, certain in vitro parameters that are important in assessing the viability of the treated red blood cell composition include, but are not limited to, measurements indicating oxygen transport activity of the red blood cells (as measured by oxygen affinity), intracellular adenosine 5′-triphosphate (ATP) levels, intracellular 2,3-diphosphoglycerate (2,3-DPG) levels, extracellular potassium levels, hemolysis or vesiculation of the red blood cells, pH, hematocrit, free hemoglobin levels, osmotic fragility of the red blood cells, deformability of the red blood cells by ektacytometry, ion homeostasis (Na⁺, K⁺ and SO₄⁻ fluxes), active cation transport (ouabain sensitive Na⁺ transport, bemetanide sensitive Na⁺, K⁺ transport), glucose consumption and lactate production.

Methods for determining ATP, 2,3-DPG, glucose, hemoglobin, hemolysis, and potassium are available in the art. See for example, Davey et al., Transfusion, 32:525-528 (1992), the disclosure of which is incorporated herein by reference. Methods for determining red blood cell function are also described in Greenwalt et al., Vox Sang, 58:94-99 (1990); Hogman et al., Vox Sang, 65:271-278 (1993); Beutler et al., Blood, Vol. 59 (1982); and Beutler, Red blood cell Metabolism, 3rd edition, Grune & Stratton, (1984) the disclosures of which are incorporated herein by reference. Extracellular sodium and potassium levels may be measured using a Ciba Corning Model 614 K⁺/Na⁺ Analyzer (Ciba Corning Diagnostics Corp., Medford, Mass.). The pH can be measured using a Ciba Corning Model 238 Blood Gas Analyzer (Ciba Corning Diagnostics Corp.).

These measurements are compared to an untreated control red blood cell composition to determine whether the function of the treated composition has been significantly reduced. In one embodiment, a red blood cell composition having reduced risk of being infectious and reduced immunogenicity will have extracellular potassium of no more than 3 times and more preferably no more than 2 times the level measured in an untreated control red blood cell composition 1 day after treatment. In another embodiment, hemolysis of the treated red blood cell composition is less than 5% after treatment and after up to 42 days storage at 4° C. In another embodiment, hemolysis of the treated red blood cell composition after storage at 4° C. is less than 3% after 28 days, more preferably less than 2% after 35 days, more preferably less than or equal to about 0.8% after 35 days, more preferably 42 days. In another embodiment, the treated red blood cell composition will have intracellular ATP levels that are within 50%, more preferably 25%, and more preferably 10%, of the level of the untreated control composition directly after treatment, preferably after 28 days storage at 4° C., more preferably after 42 days storage at 4° C. In another embodiment, the treated red blood cell composition will have intracellular 2,3-DPG levels that are within 90%, more preferably 50%, and more preferably 25%, of the level of the untreated control composition directly after treatment, preferably after 7 days storage at 4° C.

In another embodiment of the present invention, it is contemplated that the treated red blood cells have significantly reduced immunogenicity (i.e. reduced immune reaction) relative to an untreated red blood cell control or a red blood cell treated only to inactivate pathogens. Certain in vitro assays, known to those skilled in the art, may be carried out to assess immunogenicity of a treated red blood cell relative to an untreated control. In vitro assays include, but are not limited to, ABO reactivity agglutination, measurement of red blood cell aggregation as a function of antibody added to the composition, reactivity to minor antigens, ELISA assay to measure direct binding of antibody, and analysis of bound fluorescent antibody to assess levels of modification by immune masking compounds (e.g. Example 9).

As an example of ABO reactivity assessment, a composition containing treated red blood cells is reacted with serum containing a suitable antibody (e.g. treated type A red blood cells would be reacted with serum containing anti-A antibodies) and agglutination of the red blood cells is observed. The reaction is repeated with serially diluted aliquots of the antibody containing serum until no agglutination is observed. An embodiment of the present invention contemplates a treated red blood cell composition which requires at least a 2³ fold lesser dilution, preferably at least a 2⁵ fold lesser dilution, more preferably at least a 2⁷ fold lesser dilution of the antiserum relative to that required for an untreated control red blood cell composition in order to observe lack of agglutination. Another example is an ELISA assay to measure the binding of antibodies to the red blood cell antigens using an anti-human IgG conjugated to alkaline phosphatase. Another embodiment of the present invention contemplates a treated red blood cell composition which is Rh positive (i.e. has the D antigen) in which binding of an anti-D antibody to the treated red blood cells relative to an untreated Rh positive red blood cell control, as measured by such an ELISA assay, is reduced by at least 75%, preferably at least 90%, preferably at least 95%, and most preferably more then 99%. The immunogenicity of minor antigens, which are generally implicated in alloimmunization, can also be tested in vitro. The established system rates the reaction on a scale of 0-4⁺, 0 being no reaction, 4⁺ being the highest level of agglutination [Walker et al., AABB Technical Manual, 10th Ed., pp 528-537 (1990)]. Minor antigens that have been implicated in alloimmunization include Jk^{a}, E, K, Bg, Lu^{a}, P₁, D, Sd^{a}, Fy^{a}, M, Yk^{a}, A₁, Le^{a}, Kp^{a}, C, e, and I [Heddle et al., Brit. J. Hemat. 91:1000-5 (1995)]. Another embodiment of the present invention contemplates a composition containing treated red blood cells which shows in vitro reactivity in this assay for anti-D, anti-Jk^{a}, anti-E, anti-C, anti-e, or anti-K of less than or equal to 2⁺, preferably less than or equal to 1⁺, most preferably 0 on this rating scale of 0-4⁺.

In one embodiment of the present invention, the treated red blood cells have reduced immunogenicity such that infusion into an ABO mismatched recipient (e.g. treated donor type A red blood cells infused into a type B recipient) would not result in an acute hemolytic reaction. In another embodiment, infusion of treated donor red blood cells into a recipient who is allosensitized (i.e. has developed antibodies to a minor antigen present on the untreated donor red blood cell, or an antigen resulting from pathogen inactivation treatment) would not result in an immune reaction to the alloantigens and rapid clearance of the red blood cells. It is also possible to use certain in vivo assays to assess the immunogenicity of a treated red blood cell relative to an untreated control. In vivo survival studies may be done to assess immunogenicity, for example by assaying in vivo survival of treated sheep red blood cells in mice, or preferably in vivo survival in a model species, such as canines, of transfused red blood cells wherein the untreated red blood cells would elicit an immune response (i.e. antigen mismatched red blood cells). An embodiment of the present invention is one in which the in vivo survival of treated dog red blood cells is substantially increased over that of an untreated dog red blood cells from a donor which is antigen mismatched to the recipient. In general, substantially increased survival would be a survival of a red blood cell treated to reduce immunogenicity of approximately 2×, more preferably 5× and more preferably 10× the survival of an untreated red blood cell control.

The present invention also contemplates methods of use comprising the above mentioned compositions and medicaments. An example of a method of use comprises the delivery of the composition or medicament into an individual in need of a red blood cell transfusion. Another embodiment contemplates a red blood cell processing system comprising compositions or medicaments as described above and a suitable container for storing the red blood cell composition wherein the red blood cell composition is suitable for delivery to an individual. In a preferred embodiment, the container is a blood bag.

The present invention further contemplates methods of producing a red blood cell composition that has a significantly reduced risk of being infectious and has significantly reduced immunogenicity. In one embodiment, the method comprises the steps of 1) treating a red blood cell composition in order to substantially inactivate a pathogen that may be present in the red blood cell composition and 2) subsequently treating the red blood cell composition in order to substantially mask antigens so as to significantly reduce the immunogenicity of the red blood cell composition such that the treated red blood cell composition remains functional for its intended use, for example, in vivo use. In one embodiment, the process can be carried out from step 1 to step 2 without additional manipulation of the sample, i.e. without dilution, washing, addition of buffers, quenching of the reaction, compound removal, etc. In another embodiment, the treated red blood cell composition is additionally washed or treated to reduce the level of any unwanted reagents of the process or side products of these reagents, or unwanted by-products of the process.

In another embodiment, the invention comprises an ex vivo method of treating a red blood cell composition comprising contacting the red blood cell composition with a compound that substantially inactivates a pathogen that may be present in the composition, under conditions that result in substantial inactivation of the pathogen present, if any; and contacting the red blood cell composition with a compound that binds to the red blood cells and substantially masks red blood cell antigens under conditions that significantly reduce the immunogenicity of the red blood cells such that transfusing the red blood cell composition into an antigen mismatched animal would result in a reduced immune reaction compared to the immune reaction of transfusing an untreated red blood cell composition.

In another embodiment, the method comprises the steps of 1) adding an inactivation compound to a red blood cell composition, under conditions such that the inactivation compound reacts with a pathogen that may be present in the red blood cell composition and substantially inactivates the pathogen and 2) subsequently adding a non-immunogenic compound to the red blood cell composition that covalently binds to the red blood cells in the composition, thereby masking antigens, under conditions that significantly reduce the immunogenicity of the red blood cells in the composition, such that the resulting treated red blood cell composition remains functional for its intended use, such as for transfusion. In one embodiment, the process can be carried out from step 1 to step 2 without additional manipulation of the sample, i.e. without dilution, washing, addition of buffers, etc. In a preferred embodiment, prior to adding the non-immunogenic compound that covalently binds to red blood cells, the red cells are washed with a suitable buffer such that the extracellular pH is optimal for reaction of the non-immunogenic compound with the red blood cells. A preferred buffer is one which results in an extracellular pH of approximately 8-9 and maintains buffering capacity at that pH. In another embodiment, the treated red blood cell composition is additionally washed or treated to reduce the level of any unreacted inactivation compound or antigen masking compound or any unwanted side products of these reagents, or unwanted by-products of the process. It is understood that the reaction of the inactivation compound with a pathogen may be aided by an external energy source, for example, by irradiation with an appropriate light source.

In another embodiment, the method comprises providing a red blood cell composition suspected of containing a bacterium, wherein said bacterium, if present, is reacted with an antigen masking compound such that the bacterium is more infectious (i.e. the bacterium is less immunogenic and therefor more harmful to an individual) than a bacterium that is not reacted with the antigen masking compound, contacting the red blood cell composition with a compound that substantially inactivates the bacterium that may be present in the composition, under conditions that result in substantial inactivation of the bacterium present, if any; and contacting the red blood cell composition with a sufficient amount of the antigen masking compound such that the antigen masking compound binds to the red blood cells and substantially masks red blood cell antigens under conditions that significantly reduce the immunogenicity of the red blood cells such that transfusing the red blood cell composition into an antigen mismatched animal would result in a reduced immune reaction compared to the immune reaction of transfusing an untreated red blood cell composition, wherein the red blood cells are suitable for use in vivo.

In another embodiment, the method comprises adding a compound having a nucleic acid binding ligand and a nucleic acid reactive effector group to a red blood cell composition suspected of containing a pathogen, said compound reaching a final concentration sufficient to inactivate substantially all of said pathogen, to create a mixture, and incubating said mixture until substantially all of said pathogen is inactivated and further adding a compound having a polyethylene glycol group attached to a coupling group, to create another mixture, and incubating said mixture to create an incubated mixture under conditions wherein said polyethylene glycol group is covalently attached to the red blood cells to a level in which any red blood cell antigens are substantially masked from recognition by an allogeneic immune system, the overall process resulting in a red blood cell composition which has a significantly reduced risk of being infectious, has significantly reduced immunogenicity, remains functional, and is suitable for in vivo use. In one embodiment of this method, a portion of the coupling group remains between the polyethylene glycol group and the red blood cell while in another embodiment, the coupling group is eliminated, resulting in direct attachment of the polyethylene group to the red blood cell. In another embodiment, the addition of the polyethylene glycol group can be carried out without additional manipulation of the sample, i.e. without dilution, washing, addition of buffers, etc. In another embodiment, the treated red blood cell composition is additionally washed or treated to reduce the level of any unreacted nucleic acid binding mustard or polyethylene glycol compound or any unwanted side products of these reagents, or unwanted by-products of the process.

In another embodiment, the method comprises the steps of 1) treating a red blood cell composition in order to substantially mask antigens so as to significantly reduce the immunogenicity of the red blood cell composition and 2) subsequently treating the red blood cell composition in order to substantially inactivate a pathogen that may be present in the red blood cell composition such that the treated red blood cell composition remains functional, and is suitable for in vivo use. In another embodiment, the process can be carried out from step 1 to step 2 without additional manipulation of the sample, i.e. without dilution, washing, addition of buffers, etc. In another embodiment, the treated red blood cell composition is additionally washed or treated to reduce the level of any unwanted reagents of the process or side products of these reagents, or unwanted by-products of the process.

In another embodiment, the method comprises the steps of 1) adding a non-immunogenic compound to the red blood cell composition that covalently binds to the red blood cells in the composition, thereby masking antigens, under conditions that significantly reduce the immunogenicity of the red blood cells in the composition and 2) subsequently adding an inactivation compound to the red blood cell composition, under conditions such that the inactivation compound reacts with a pathogen that may be present in the red blood cell composition and substantially inactivates the pathogen, such that the resulting treated red blood cell composition remains functional, and is suitable for in vivo use. In another embodiment, the process can be carried out from step 1 to step 2 without additional manipulation of the sample, i.e. without dilution, washing, addition of buffers, etc. In another embodiment, the treated red blood cell composition is additionally washed or treated to reduce the level of any unreacted inactivation compound or antigen masking compound or any unwanted side products of these reagents, or unwanted by-products of the process. In a preferred embodiment, prior to adding the non-immunogenic compound that covalently binds to red blood cells, the red cells are washed with a suitable buffer such that the extracellular pH is optimal for reaction of the non-immunogenic compound with the red blood cells. A preferred buffer is one which results in an extracellular pH of approximately 8-9 and maintains buffering capacity at that pH. It is understood that the reaction of the inactivation compound with pathogens may be aided by an external energy source, for example, by irradiation with an appropriate light source.

In another embodiment, the method comprises adding a compound having a polyethylene glycol group attached to a coupling group to a red blood cell composition suspected of containing a pathogen, to create a mixture, and incubating said mixture to create an incubated mixture under conditions wherein said polyethylene glycol group is covalently attached to the red blood cells to a level in which any red blood cell antigens are substantially masked from recognition by an allogeneic immune system and further adding a pathogen inactivating compound having a nucleic acid binding ligand and a nucleic acid reactive effector group, said compound reaching a final concentration sufficient to inactivate substantially all of said pathogen, to create a mixture, and incubating said mixture to create an incubated mixture wherein substantially all of said pathogen is inactivated, the overall process resulting in red blood cell composition which has a significantly reduced risk of being infectious, has significantly reduced immunogenicity, remains functional, and is suitable for in vivo use. In one embodiment of this method, a portion of the coupling group remains between the polyethylene glycol group and the red blood cell while in another embodiment, the coupling group is eliminated, resulting in direct attachment of the polyethylene group to the red blood cell. In another embodiment, the nucleic acid reactive effector group is a mustard group and the addition of the nucleic acid binding mustard compound can be carried out without additional manipulation of the sample, i.e. without dilution, washing, addition of buffers, etc. In another embodiment, the treated red blood cell composition is additionally washed or treated to reduce the level of any unreacted nucleic acid binding mustard or polyethylene glycol compound or any unwanted side products of these reagents, or unwanted by-products of the process.

In another embodiment, the method comprises concurrently treating a red blood cell composition with the reagents described above under conditions which substantially mask antigens so as to significantly reduce the immunogenicity of the red blood cell composition and to substantially inactivate a pathogen that may be present in the red blood cell composition such that the treated red blood cell composition remains functional and suitable for in vivo use. In another embodiment, the treated red blood cell composition is additionally washed or treated to reduce the level of any unwanted reagents of the process or side products of these reagents, or unwanted by-products of the process.

In another embodiment, the method comprises concurrently adding a non-immunogenic compound to the red blood cell composition that covalently binds to the red blood cells in the composition, thereby masking antigens, under conditions that significantly reduces the immunogenicity of the red blood cells in the composition and adding an inactivation compound to the red blood cell composition, under conditions such that the inactivation compound reacts with a pathogen that may be present in the red blood cell composition and substantially inactivates the pathogen, so that the resulting treated red blood cell composition remains functional for its intended use, such as for transfusion. The treated red blood cell composition can be additionally washed or treated to reduce the level of any unreacted inactivation compound or non-immunogenic compound or any unwanted side products of these reagents, or unwanted by-products of the process. It is understood that the reaction of the inactivation compound with pathogens may be aided by an external energy source, for example, by irradiation with an appropriate light source, without interfering with the concurrent reaction of the non-immunogenic compound to mask the antigens.

In some embodiments of the methods of the invention, a portion of the coupling group remains between the polyethylene glycol group and the red blood cell while in other embodiments, the coupling group is eliminated, resulting in direct attachment of the polyethylene group to the red blood cell.

It is to be recognized that the step or steps of inactivating pathogens and masking of antigens may be quenched to reduce any unwanted side products of the reactions in all embodiments discussed above and the quenching is not limited to a single step. In one embodiment, the pathogen inactivation step is done in combination with a treatment to quench unwanted side products, wherein a quencher may be added before, simultaneously with, or after treatment to inactivate pathogens. In another embodiment, the antigen masking step is done in combination with a treatment to quench unwanted side products, wherein a quencher may be added before, simultaneously with, or after treatment to reduce the immunogenicity of the red blood cells. It is to be recognized that the quenching treatment may or may not be the same for the pathogen inactivation step and the antigen masking step and may be required for either or both steps, or in the case where the processes are carried out concurrently. It is also an embodiment of the invention that the quenching treatment be done at any time in the process yet be effective at quenching both steps of the process, or both processes in the case where they are done concurrently.

It is also recognized that treatment to reduce the level of unwanted reagents or by-products as discussed in all embodiments above is not limited to any particular step of the process and is not limited to a single step. In one embodiment, a compound reduction device is used following the step or steps of inactivating pathogens and masking of antigens to reduce the level of unwanted compounds. In further embodiments, a compound reduction device is used after each step to reduce the level of unwanted compounds, and further the compound reduction devices used after each step may be of different compositions.

It is also recognized that in all embodiments discussed above, the red blood cell composition may be treated, e.g. by washing, prior to treatment to mask the antigens of the red blood cells, in order to remove excess biological fluid(s) which contains proteins and other macromolecules which may compete with the red blood cells for the immune masking compounds. This may be necessary to ensure uniform reaction of the immune masking compound with the red blood cells to reproducibly prepare a red blood cell composition which has sufficiently reduce immunogenicity.

IV. Selecting Compounds and Methods for Inactivation of Pathogens and Preparation of Non-immunogenic Red Blood Cells.

In order to evaluate compounds and methods to decide if they would be useful in the present invention, three important properties should be considered: the compound and method's ability to inactivate pathogens, the compound and method's effect on the immunogenicity of the red blood cell, and the effect of the compounds and methods on the functioning of the red blood cell composition for its intended use. Screening techniques to measure these parameters are known to those of skill in the art.

A screening technique used to evaluate the compounds of the present invention for their ability to inactivate pathogens is a bacteriophage screen; an assay that depends on nucleic acid binding of test compounds. A screen of this type, an R17 screen, is described in detail in Example 1, below. The R17 bacteriophage screen is believed to be predictive of HIV inactivation efficiency, as well as the efficiency of compounds against many other viruses. A similar screening assay could be done with MS2 bacteriophage as well. Additional inactivation studies to further assess the compounds and methods of the present invention are described in Examples 2-3.

Screening techniques for immunogenicity of cells include those utilizing antibodies to recognize cell surface antigens. For example, a screening technique used to evaluate the compounds of the present invention for their ability to reduce the immunogenicity of the red blood cells is to perform separate agglutination tests using anti-A, anti-B, and anti-D antibodies; an assay that determines the immunogenicity of the red blood cells. A screen of this type is described in detail in Example 4, below. The particular compounds and methods of the present invention can be optimized for the maximum reduction in the immunogenicity of the A, B, and D antigens using this assay. Those compounds and methods which result in at least a 2³ fold reduction in the dilution of the anti-A or anti-B antiserum needed to see no agglutination relative to the dilution needed for an untreated control red blood cell sample and those compounds and methods which result in a score of 1⁺ or lower for anti-D antiserum are expected to be reasonable candidates for preparation of red blood cell compositions of the present invention.

Additional screening techniques may utilize a detection label such as radioactive or fluorescent labeled compounds. In such assays, the amount of suitably labeled PEG on the surface of the red blood cells can be measured directly by isolation and analysis of the red blood cell membranes (ghosts) or other methods of partitioning measurement for the red blood cells. Alternatively, the amount of fluorescently labeled PEG on the surface of the red blood cells can be directly measured using a flow cytometer. The fluorescent signal can be correlated to the relative amount of fluorescent PEG used and compared to a standard curve using, for example, beads containing known amounts of fluorescently labeled molecules. An example of the measurement of the modification density of PEG modified red blood cells is given in Example 11.

Screening techniques used to evaluate red blood cell compositions and methods of the present invention include, but are not limited to, measurement of intracellular ATP, intracellular 2,3-DPG, extracellular potassium, hemolysis, osmotic fragility and oxygen transport activity as described in the examples below. Those compounds and methods, when assayed relative to an untreated control sample, which do not vary significantly from the control sample (i.e. are within an acceptable range according to current standards of blood banking practice) are expected to be reasonable candidates for preparation of red blood cell compositions of the present invention.

### EXPERIMENTAL

### EXAMPLE I

Determination of R17 Inactivation by Compounds and Methods of the Present Invention.

This example describes the assay for measuring the inactivation of the bacteriophage R17 by the compounds and methods of the present invention. It is an intent of the assay to assess inactivation at various steps in the process using the compounds of the present invention. In particular, the compounds and methods for inactivation are to be assayed separately from the compounds and methods for masking the immunogenicity of the red blood cells. The overall process resulting in the less infectious, less immunogenic red blood cell composition will be assayed as well. It is important to assess these separately as there is concern of the possibility that masking the immunogenicity of the red blood cells might also mask the immunogenicity of the R17. It is not known whether this will itself inactivate the R17 and it is a goal of this example to make that determination.

The assay measures the ability of the bacteriophage to subsequently infect bacteria and inhibit their growth. The phage is grown up in Hrf 3000 bacteria (R17 and Hrf 3000 can be obtained from American Tissue Culture Collection (ATCC), Washington, D.C.). First, the R17 stock virus is diluted (approximately 10.9 logs/ml in LB broth) 1:20 in Adsol (R17-Adsol). Then a 30% hematocrit red blood cell concentrate in R17-Adsol mixture is prepared by spinning down red blood cells from whole blood (Sacramento Blood Center, CPDA-1 collected) and resuspending 3.5 ml of the red blood cell pellet in 7.0 ml R17-Adsol. The hematocrit can be measured on a model F800 Sysmex cell counter (Toa Medical Electronics, Kobe, Japan). One ml aliquots of this mixture are used to assay the compounds for inactivation. The volumes or red blood cell pellet and R17-Adsol can be adjusted as needed. Samples of pathogen inactivating compound are dissolved in DMSO or Adsol to a desired concentration and added in aliquots of 50 μl or less to the aliquots of the 30% hematocrit red blood cell composition to the desired final concentration. In addition, an appropriate volume of DMSO or Adsol is added to bring the total volume (compound +added DMSO or Adsol) added to 50 μl. For a positive control, 50 μl of DMSO or Adsol is added to the red blood cell composition. These are allowed to stand at room temperature for at least 1 hour. In the case of inactivation compounds that require external activation energy, e.g. irradiation, the appropriate activation is done instead of the 1 hour incubation. The samples are then titered by an R17 phage assay by doing sterile dilutions of 0. 1 ml phage solution to 0.4 ml of LB broth. They are then diluted 0.02 ml with 0.5 ml of LB broth (1:25) and subsequently serially diluted (1:25) into LB broth. To each diluted sample, 0.05 ml of Hrf 3000 bacteria cultured overnight is added along with 3 ml of molten LB top agar. The mixture is poured onto LB broth plates, which are placed in a 37° C. incubator after the top agar hardens. Plaques are counted after overnight incubation and the titer of the phage remaining is calculated based on dilution factors.

In order to examine the effect of compounds for masking the immunogenicity of the red blood cells as well as the effect of the entire method for preparation of the treated red blood cell composition, the solution of R17-Adsol is treated appropriately and subsequently plated as described above. Appropriate controls are run substituting adding the appropriate solution without inactivation or antigen masking compound in order to assess the titer of inactivation. It is also possible to substitute other additive solutions for Adsol in this assay.

### EXAMPLE 2

Determination of HIV Inactivation by Compounds and Methods of the Present Invention.

Cell associated HIV in TC Medium (Popovic et al., Science, 224:497 (1984): H9-IIIb cells are suspended in Tissue Culture Medium to provide a suspension with a determined titer measured as plaque forming units/mL. To 2 mL aliquots of the test medium in 15 mL conical tubes is added a sufficient amount of pathogen inactivation compound solution to achieve the desired concentration of active material. The suspensions are immediately mixed by fully pipetting several times, then vortexing briefly. The samples are incubated at ambient temperature for 2-4 h and then centrifuged (in the case of inactivation compounds which require external activation energy, e.g. irradiation, the appropriate activation is done instead of the 2-4 hour incubation). The pellets are resuspended in 1 mL of plaque assay diluent, then quickly frozen at −80° C. and titrated by a microplaque assay. (Hanson et al., J. Clin. Micro., 28:2030 (1990)).

Cell-associated HIV in packed red blood cells (PRBC): For assays run in PRBC, the packed cells are prepared by taking whole blood (Sacramento Blood Center, CPDA-1 collected) of measured hematocrit and centrifuging at 3800 rpm (4097× g) for 6 minutes. The supernatant plasma is removed and the volume removed is measured. Adsol solution is added to the red blood cells to give a PRBC with a 60% hematocrit. The plasma concentration in this preparation will be 15-20%. The HIV9-IIIb cells are added to the Adsol prior to dilution of the centrifuged cells. The resultant suspension is mixed by fully pipetting all the material, including a sufficient amount of pathogen inactivating compound solution. Upon completion of incubation or external activation of the test compound, the samples are diluted with 3 mL of a 1:1 plasma:DMEM solution containing 5 mL of heparin. The infected cells are then isolated using a ficol-hypaque gradient, resuspended in 1 mL of the diluent, and frozen for later titration.

Cell-free HIV in PRBC: The protocol is similar to that described above, except that cell-free HIV is added directly to the PRBC after preparation. After incubation, the medium is centrifuged and the supernatant is frozen for later titration.

In order to examine the effect of compounds for masking the immunogenicity of the red blood cells as well as the effect of the entire method for preparation of the treated red blood cell composition, the solution of HIV are treated appropriately and subsequently titrated as described above. In this scenario, appropriate controls are run substituting adding the appropriate solution without inactivation or antigen masking compound in order to assess the titer of inactivation. It is also possible to substitute other additive solutions for Adsol in this assay.

### EXAMPLE 3

Determination of Yersinia enterocolitica Inactivation by Compounds and Methods of the Present Invention.

Yersinia enterocolitica (California Department of Health Services, Microbial Disease Laboratory, Berkeley, Calif.) is cultured in LB-broth at 37° C. overnight on a shaker at 180 rpm. To measure the titer, the optical density is measured of a 1:100 dilution in Adsol (OD₆₁₀=0.2 at 10⁸ bacterial/mL). The bacterial stock is then diluted 1:100 into saline or PRBC to provide the test medium which is aliquoted (1 mL) into 2 mL o-ring sterile tubes. The PRBC is prepared as described above in Example 2.

To each tube is added a sufficient amount of the pathogen inactivating solution to provide the appropriate concentration of test compound. Each sample is quickly mixed by fully pipetting the mixture several times. It is then incubated for two hours at ambient temperature or treated for external activation, then plated out on LB-agar starting with 100 μL sample starting at 10′ dilution and continuing dilutions to 10⁻⁸. The plates are incubated overnight at 37° C. and the colonies are counted. The difference between the titer of the untreated test medium and that of a treated sample provides the log kill for the compound at that concentration. The detection limit is 10 bacteria/mL, but this limit can be reduced to 1 bacteria/mL by assaying 10 plates per sample.

In order to examine the effect of compounds for masking the immunogenicity of the red blood cells as well as the effect of the entire method for preparation of the treated red blood cell composition, the solution of Yersinia is treated appropriately and subsequently plated as described above. In this scenario, appropriate controls are run substituting adding the appropriate solution without inactivation or antigen masking compound in order to assess the titer of inactivation. It is also possible to substitute other additives for Adsol in this assay.

### EXAMPLE 4

Determination of Agglutination Reaction of Red Blood Cells of the Present Invention With Anti-A, Anti-B, and Anti-D Antisera.

The process of inactivation of pathogens and antigen masking is carried out under appropriate conditions on CPDA-1 collected red blood cells. Polyethylene glycol derivatives may be purchased from Shearwater Polymers (Huntsville, Ala.). Agglutination reactions of the treated red blood cells are assayed by standard techniques as described in Walker et al., AABB Technical Manual, 10th Ed., pp. 528-537 (1990). The agglutination reaction is assessed on serially diluted samples. The dilution level at which agglutination no longer is observed is recorded for treated red blood cells compared to untreated red blood cells. This assay is carried out using type A red blood cells and anti-A antibody or type B red blood cells and anti-B antibody. The processing of the red blood cells with respect to antigen masking can be optimized in part based on this assay.

Similar assays can be done using Rh positive red blood cells and anti-D antiserum. In this assay, the agglutination will be scored as described in the AABB technical manual. The treated red blood cells will be compared to an untreated control sample to assess ability of the process to mask the D antigen.

### EXAMPLE 5

Assessment in Vitro of Red Blood Cell Function After Processing of Red Blood Cells.

The intracellular adenosine-5′-triphosphate (ATP), intracellular 2,3-diphosphoglyceric acid (2,3-DPG), extracellular potassium and hemolysis levels are readily assessed following processing of the red blood cells with compounds and methods of the present invention. The results are compared to untreated control samples to assess whether the treated red blood cells are suitable for their intended use, such as transfusion. Intracellular ATP and 2,3-DPG are measured using a Sigma ATP Kit or 2,3-DPG kit respectively (Sigma, St. Louis, Mo.). The ATP kit was used following Sigma procedure No. 366-UV hereby incorporated by reference. Extracellular potassium levels can be measured using a Ciba Corning 614 K⁺/Na⁺ Analyzer (Ciba Corning Diagnostics Corp., Medfield, Mass.). The appropriate red blood cell composition is directly sampled by the analyzer.

### EXAMPLE 6

Evaluation of the Oxygen Affinity of the Processed Red Blood Cells.

Following the processing of red blood cells with the compounds and methods of the present invention, oxygen affinity of the red blood cell samples is measured with a Hemox analyzer. The Hemox analyzer is pre-equilibrated at 37° C. Fifty μL of the red blood cell sample is mixed with 3.97 mL Hemox buffer solution (TCS Scientific Corp., New Hope, Pa.), containing 20 μL of 20% Bovine Serum Albumin (TCS Scientific Corp.) and 10 μL anti-foaming reagent (TCS Scientific Corp.) before transferring into the Hemox Analyzer cuvette. After the diluted sample is drawn into the cuvette, the temperature of the mixture is equilibrated with stirring for 8 minutes at 37° C. Subsequently, the diluted sample is fully oxygenated by exposure to air for 8 minutes. The instrument is calibrated for the partial pressure reading and the degree of hemoglobin saturation for each sample. The log ratio of the solution absorption at 560 to the absorption at 570 nm is recorded on the Y-axis while the partial pressure of oxygen (pO₂) obtained from a Clark electrode is recorded on the X-axis. The X-axis is calibrated by assigning values of 0 and the maximum calculated pO₂ for the day to readings obtained from 100% nitrogen and 100% air. The Y-axis is calibrated by assigning values of 0 and 1 to readings obtained from hemoglobin equilibrated under nitrogen or oxygen, respectively. For each sample an oxygen affinity curve is obtained by lowering the pO₂ through the introduction of nitrogen to the space above the liquid sample and measuring the percent of oxygen saturation of hemoglobin. The numerical data is converted to a graph of the oxygen affinity curve through the use of the computer program Kaleidagraph 3.0.5 (Synergy Software, Reading, Pa.) and the P₅₀ is determined from the half point of the curve. Measurements can be made on treated samples and compared to measurements of untreated control samples.

### EXAMPLE 7

Evaluation of the Osmotic Fragility of the Processed Red Blood Cells.

The osmotic fragility of samples is measured for red blood cells processed with compounds and methods of the present invention and compared to untreated control samples. Reagent is prepared at 0.1, 0.2, 0.3, 0.35, 0.4, 0.45, 0.5, 0.55, 0.6, 0.65, 0.75, and 0.9% PBS (1.0% PBS is 9g NaCl, 1.365g Na₂HPO₄, and 0.186g NaH₂PO₄ to a final volume of 1 liter in water). A 10 μL aliquot of red blood cell sample is added to 1.0 mL of each of these solutions, mixed gently and incubated at room temperature for 30 minutes. After incubation, the sample is mixed gently and centrifuged for 2 minutes at 2,000× g. A spectrophotometer is zeroed with water and the absorption of the supernatant of the sample is measured at 540 nm. The % lysis is calculated using the following formula, in which the 0.9% PBS sample is considered background lysis and the 0.1% PBS sample is considered to be 100% lysis.

% lysis=(A₅₄₀−0.9%A₅₄₀)÷(0.1%A₅₄₀%−0.9%A₅₄₀)×100

The % lysis is plotted as a function of the % PBS and the plots are compared for treated red blood cells and untreated control red blood cells.

### EXAMPLE 8

Synthesis of Frangible β-alanine, N-(acridin-9-yl), 2-[bis(2-chloroethyl) amino]ethyl ester.

Step A. β-Alanine, N-(tert-butoxycarbonyl), 2-[bis(2-hydroxyethyl)amino]ethyl ester

To a stirred solution of N-(tert-butoxycarbonyl)-β-alanine (20.3 g, 107 mmol) and 4-methylmorpholine (13.0 mL, 12.0 g, 119 mmol) in dry THF (200 mL) at −15° C. under N₂ was added isobutyl chloroformate (13.9 mL, 14.6 g, 107 mmol) resulting in the immediate formation of a white precipitate (4-methylmorpholine.HCl). The reaction mixture was stirred at −15° C. for 5 min. followed by the transfer of the reaction mixture to a flask containing a stirred solution of triethanolamine (48.3 g, 324 mmol) in dry THF (150 mL) at −15° C. The reaction mixture was allowed to warm to 23° C. and stirred for an additional 1.5 h followed by removal of the precipitate by vacuum filtration. The THF was then removed in vacuo from the filtrate and the remaining viscous yellow oil was partitioned between water (500 mL) and EtOAc (5×150 mL). The combined organic layers were dried over Na₂SO₄. Removal of solvent in vacuo gave 25.8 g (75%) of the desired product, β-alanine, N-(tert-butoxycarbonyl), 2-[bis(2-hydroxyethyl)amino]ethyl ester, as a pale yellow oil. ¹H NMR: d 5.32 (br s, 1H), 4.18 (t, J=5.4 Hz, 2H), 3.58 (t, J=5.1 Hz, 4H), 3.37-3.23 (m, 2H), 2.80 (t, J=5.4 Hz, 2H), 2.69 (t, J=5.1 Hz, 4H), 2.51 (t, J=6.0 Hz, 2H), 1.41 (s, 9H). The hydroxyl protons were not observed. ¹³C NMR: d 173.0, 156.4, 79.8, 63.3, 60.2, 57.3, 54.1, 36.7, 35.3, 28.8.

Step B. β-Alanine, N-(tert-butoxycarbonyl), 2-[bis(2-tert butyldimethylsilyloxyethyl)amino]ethyl ester

A stirred solution of the β-alanine, N-(tert-butoxycarbonyl), 2-[bis(2-hydroxyethyl)arnino]ethyl ester from step A (22.7 g, 70.9 mmol) and imidazole (11.1 g, 163 mmol) in acetonitrile (70 mL) under N₂ was cooled to 0° C. Tert-butyldimethylsilyl chloride (534 mg, 3.54 mmol) was then added and the reaction mixture was stirred for an additional 5 min. at 0° C. The reaction mixture was allowed to warm to 23° C. and stirred for 2 h followed by removal of the resultant white precipitate (imidazole.HCl) by vacuum filtration. The acetonitrile was removed in vacuo from the filtrate and the remaining material was partitioned between saturated brine (600 mL) and EtOAc (3×200 mL). The combined organic layers were dried over Na₂SO₄. Removal of solvent in vacuo gave 35.2 g (90%) of the desired product, β-alanine, N-(tert-butoxycarbonyl), 2-[bis(2-tert-butyldimethylsilyloxyethyl)amino]ethyl ester, as a yellow oil. ¹H NMR: d 5.29 (br s, 1H), 4.14 (t, J=6.0 Hz, 2H), 3.65 (t, J=6.3 Hz, 4H), 3.37 (apparent q, 2H), 2.85 (t, J=6.0 Hz, 2H), 2.71 (t, J=6.3 Hz, 4H), 2.49 (t, J=5.9 Hz, 2H), 1.42 (s, 9H), 0.88 (s, 18H), 0.03 (s, 12H); ¹³C NMR: d 172.7, 156.3, 79.7, 63.3, 62.4, 57.7, 54.3, 36.7, 35.3, 28.9, 26.4, 18.7, −4.9.

Step C. β-Alanine, 2-[bis(2-tert-butyldimethylsilyloxyethyl)amino] ethyl ester

To a flask containing β-alanine, N-(tert-butoxycarbonyl), 2-[bis(2-tert-butyldimethylsilyloxyethyl)amino]ethyl ester from step B (3.01 g, 5.48 mmol) was added neat trifluoroacetic acid (5 mL) resulting in the evolution of CO₂ gas. The reaction mixture was stirred for 5 min. and the trifluoroacetic acid was removed in vacuo. The remaining material was partitioned between saturated NaHCO₃ (100 mL) and EtOAc (3×30 mL). The combined organic layers were dried over Na2SO4. Removal of solvent in vacuo gave 2.45 g (100%) of the desired product, β-alanine, 2-[bis(2-tert-butyldimethylsilyloxyethyl)amino]ethyl ester, as a pale yellow oil. ¹H NMR: d 4.12 (t, J=6.0 Hz, 2H), 3.63 (t, J=6.4 Hz, 4H), 2.96 (t, J=6.2 Hz, 2 H), 2.84 (t, J=6.0 Hz, 2H), 2.69 (t, J=6.4 Hz, 4H), 2.44 (t, J=6.2 Hz, 2H), 0.86 (s, 18H), 0.03 (s, 12H). The amine protons were not observed. ¹³C NMR (CDCl₃): d 173.0, 63.4, 62.6, 57.9, 54.4, 38.4, 38.1, 26.4, 18.7, −4.9.

Step D. β-Alanine, N-(2-carbomethoxyacridin-9-yl), 2-[bis(2-hydroxyethyl)amino]ethyl ester

The β-alanine, 2-[bis(2-tert-butyldimethylsilyloxyethyl)amino]ethyl ester (736 mg, 1.64 mmol) was reacted with methyl 9-methoxyacridine-2-carboxylate (669 mg, 2.50 mmol) by stirring in 10 mL of CHCl₃ for 12.5 h at room temperature. The precipitate (acridone) was then filtered off and the filtrate partitioned between saturated aqueous NaHCO₃ (100 mL) and CHCl₃ (3×35 mL). The combined organic layers were dried over Na₂SO₄ and concentrated in vacuo to give 1.61 g of viscous brown oil. Deprotection of the resultant diol was carried out by dissolving the crude intermediate in 3.0 mL of THF under N₂ and, upon cooling to 0° C., treating with HF/pyridine (1.0 mL). The solution was allowed to warm to room temperature with stirring for 1 h. The volatiles were removed in vacuo and the residue was partitioned between saturated aqueous NaHCO₃ (100 mL) and CHCl₃ (3×35 mL). The combined organic layers were dried and concentrated to give 649 mg of a brownish yellow solid. Preparative TLC (C-18, CH₃CN) gave a 20% yield of the desired diol, β-alanine, N-(2-carbomethoxyacridin-9-yl), 2-[bis(2-hydroxyethyl)amino]ethyl ester (>80% pure by HPLC); ¹H NMR: d 8.82 (s, 1H), 8.21-7.94 (m, 2H), 7.94-7.72 (m, 2H), 7.59 (apparent t, 1H), 7.23 (apparent t, 1H), 4.30-4.18 (m, 2H), 4.18-4.05 (m, 2H), 3.89 (s, 3H), 3.69-3.50 (m, 4H), 2.92-2.73 (m, 4H), 2.73-2.55 (m, 4H) The amine and hydroxyl protons were not observed.

Step E. β-Alanine, N-(2-carbomethoxyacridin-9-yl), 2-[bis(2-chloroethyl)amino]ethyl ester dihydrochloride

Conversion of β-alanine, N-(2-carbomethoxyacridin-9-yl), 2-[bis(2-hydroxyethyl)amino]ethyl ester to the dichloro compound was achieved by a method similar to that of Peck, et al. (J. Am. Chem. Soc. 1959, 81:3984). A yellow solution of the product from step D (41 mg, 0.090 mmol) in neat SOCl₂ (6 mL) was stirred at room temperature for 20 hours. The SOCl₂ was then removed in vacuo to give a yellow solid (dihydrochloride salt). The material was then partitioned between saturated NaHCO₃ (50 mL) and CH₂Cl₂ (3×20 mL). The combined organic layers were dried over Na₂SO₄. Removal of solvent in vacuo gave 35.4 mg of the dichloro compound free base as an orange gum. ¹H NMR: d 8.82 (s, 1H), 8.20-7.83 (m, 4H), 7.5 (apparent t, 1H), 7.25 (apparent t, 1H), 4.36-4.15 (m, 4H), 3.93 (s, 3H), 3.48 (t, J=6.9 Hz, 4H), 3.06-2.77 (m, 4H), 2.86 (t, J=6.9 Hz, 4H). The amine proton was not observed. ¹³C NMR: d 172.3,166.6,155.2, 146.5, 144.6, 133.1, 131.6, 128.7, 124.6, 124.3, 116.1, 114.3, 63.7, 57.2, 53.5, 52.9, 46.3, 42.5, 35.2. No other carbons were observed. The HCl salt was precipitated from CH₂Cl₂ by addition of 1 M HCl in ether to give β-alanine, N-(2-carbomethoxyacridin-9-yl), 2-[bis(2-chloroethyl)amino]ethyl ester dihydrochloride (Compound IV,) as a yellow solid (81% pure by HPLC).

β-Alanine, N-(acridin-9-yl), 2-[bis(2-chloroethyl)amino]ethyl ester dihydrochloride, (Compound V) was prepared in a similar manner. Thus using 9-methoxyacridine in place of methyl 9-methoxyacridine-2-carboxylate in Step D, the intermediate diol was obtained (7.1%) as a yellow oil (74% pure by HPLC). ¹H NMR: d 8.14 (d, J=7.5 Hz, 2H), 7.93 (d, J=8.6 Hz, 2H), 7.52 (apparent t, 2H), 7.23 (apparent t, 2H), 4.36-4.08 (m, 4H), 3.76-3.5 (m, 4H), 3.08-2.60 (m, 8 H). The amine and hydroxyl protons were not observed.

A solution of the intermediate diol (37.3 mg, 0.0793 mmol) in thionyl chloride (4.0 mL) was stirred at 23° C. for 7.5 h. The thionyl chloride was removed in vacuo to give a yellow oil. The material was dissolved in ethanol (˜4 mL) and the solvent removed in vacuo. The material was then dissolved in CH₂C₁₂ (4 mL) and solvent removed in vacuo; this step was repeated twice. The material was then triturated with hexane (3×4 mL) to give 40.0 mg (42% pure by HPLC) of the product in the form of a yellow hydroscopic glassy solid. Some of the material was converted to the free amine for analytical purposes by partitioning between saturated NaHCO₃ and CH₂Cl₂ followed by drying the combined organic layers over Na₂SO₄ and removal of the solvent in vacuo. ¹H NMR: d 8.21-8.00 (m, 4H), 7.66 (apparent t, 2H), 7.38 (apparent t, 2H), 4.26-4.12 (m, 2H), 4.12-3.98 (m, 2H), 3.43 (t, J=6.9 Hz, 4H), 2.96-2.68 (m, 8H). The amine proton was not observed.

Following the above procedure but replacing N-(tert-butoxycarbonyl)-β-alanine with N-(tert-butoxycarbonyl)-4-aminobutyric acid led to the preparation of 4-aminobutyric acid N-[(2-carbomethoxyacridin-9-yl), 2-[bis(2-chloroethyl)amino]ethyl ester dihydrochloride, Compound VI (78% pure by HPLC). ¹H NMR: d 8.89 (s, 1), 8.12 (apparent t, 2), 7.93-7.80 (m, 2), 7.59 (apparent q, 1), 7.36-7.20 (m, 1), 4.16 (t, 2, J=5.7 Hz), 4.07-3.92 (m, 2), 3.97 (s, 3), 3.46 (t, 4, J=6.9 Hz), 2.93-2.80 (m, 6), 2.60 (t, 2, J=6.5 Hz), 2.29-2.12 (m, 2). The amine proton was not observed.

### EXAMPLE 9

Flow Cytometry Analysis of Red Blood Cells to Assess Levels of Polyethylene Glycol Modification.

A unit of ABO-typed whole blood (Sacramento Blood Center, Calif.) was leukofiltered according to standard blood banking methods. The red blood cells were washed three to four times with phosphate buffered saline (PBS, comprising 150 mM phosphate pH=9.2) to eliminate plasma proteins and adjust the pH of the extracellular domain to the desired value for the reaction. The volume of the suspension was adjusted with PBS to an HCT of 40%. A solution of cyanuric chloride activated MPEG was prepared in PBS (2.3 mL, 4.3 mM) and a 1 mL aliquot of the red blood cell suspension was added to this solution. The solution was mixed with a tube inverter for 1 hour and then allowed to incubate at room temperature for 16-24 hours. Following this room temperature incubation, the solution was washed three times with blood bank saline to remove any excess MPEG and any other reaction side products. Following this wash, Adsol™, comprising 154 mM NaCl, 2.0 mM adenine, 41.2 mM mannitol, and 111.0 mM dextrose (Baxter Healthcare, Ill.), was added to a final HCT of 40%. The resulting red blood cell suspension was stored at 4° C.

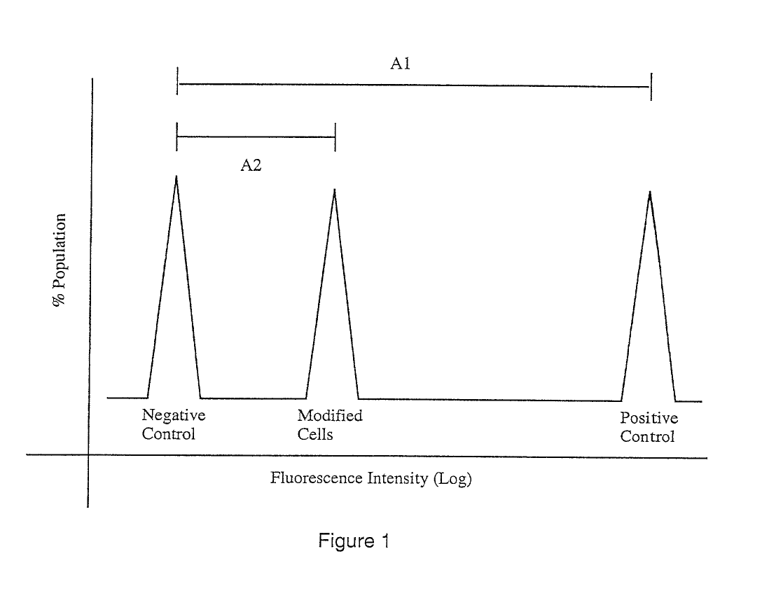

The modified cells were analyzed for their ability to bind fluorescent labeled antibody with a flow cytometry method using a FACScan™ m (Becton, Dickinson and Co., N.J.). An aliquot of cells was centrifuged and the supernatant removed. A 50 μL portion of red blood cells (approximately 1×10⁶ cells) was incubated at room temperature for 1 hour with 5 μL of an appropriate stock antibody solution (i.e. antibody would bind non MPEG modified red blood cell, e.g. anti-A FITC conjugate BRIC-145, anti-B FITC conjugate BGRL1, or anti-D FITC conjugate BRAD-3 depending on the blood type, International Blood Group Reference Laboratory, UK). The cells were subsequently washed to remove the excess of the antibody and were analyzed by flow cytometry for bound fluorescent antibodies. The level of bound fluorescent antibodies was compared to either non MPEG modified cells (positive control) or cells which were not incubated with FITC antibody (negative control). The relative degree of pegylation can be estimated based on the ratio of the population maximum fluorescence (test article-negative control)/(positive control-negative control). This is represented as A2/A1 in FIG. 1.

### EXAMPLE 10

Reaction of Red Blood Cell Composition With Tresyl MPEG or SPA MPEG and β-alanine, N-(acridin-9-yl), 2-[bis(2-chloroethyl) amino]ethyl ester.

Leukofiltered packed red blood cells (PRBC, 60% hematocrit) containing a suitable additive solution such as Erythrosol (0.782 g/100 mL sodium citrate dihydrate, 0.073 g/100 mL monosodium phosphate dihydrate, 0.303 g/100 mL disodium phosphate dihydrate, 0.022 g/100 mL adenine, 0.774 g/100 mL mannitol) is mixed with a 20 mL solution of 4.5% dextrose monohydrate containing 30 mg of β-alanine, N-(acridin-9-yl), 2-[bis(2-chloroethyl) amino]ethyl ester and 184 mg of glutathione. The resulting solution is transferred to a reaction container and incubated for 12-20 hours at 19-25° C. The solution is then hard spun to an 80-90% hematocrit, washed twice with pH=8Hepes buffer (150 mM Hepes, 50 mM NaCl, 75 mM dextrose) and subsequently diluted to a hematocrit of 40% into a Hepes buffer solution containing either Tresyl MPEG or SPA MPEG at an appropriate concentration. The solution is thoroughly mixed and then incubated at or below room temperature. After a suitable incubation period, the red blood cells are spun and the hematocrit adjusted to 60% with an appropriate additive solution. This final solution is optionally transferred to a blood bag containing an adsorption media to reduce the levels of excess reagents and byproducts. The final solution is then characterized with respect to red blood cell function and immunogenicity of the red blood cells as described in the preceding examples. The amount of MPEG modification (MPEG per red blood cell) can also be determined by the method of Example 11 below.

### EXAMPLE 11

### Measurement of Modification Density for Pegylated RBC

RBC were prepared for pegylation as per example 10. In the pegylation step the activated MPEG was replaced with a mixture of the activated MPEG plus FITC labeled activated MPEG of the same kind, which bears a fluorescent label on the other end. Alternatively, the activated MPEG could be modified with other detectable labels, such as a radioactive isotope. A 50:50 mixture was used for this experiment. The reaction was allowed to proceed for 2 hrs at RT and the cells were subsequently washed to remove the reaction side products. Red blood cell concentrate (200 μL) was subsequently used to make ghost membranes through controlled lysis with chilled hypotonic lysis buffer (1600 μL) (7.5 mM sodium phosphate, 1 mM NaEDTA, pH 7.5). The resulting ghosts isolated through centrifugation (14000× g; 2 min) were washed 4 times with chilled lvsis buffer and then taken up in a 250 μl. volume of the same buffer. SDS was added to the suspension (final [SDS]=1%) in order to achieve complete dissolution of the membranes. The resulting solution was further diluted 5 fold in lysis buffer and then analyzed for fluorescence (λ_{exc}=490 nm, λₑₘₘ=525 mn). The amount of fluorescent label was quantitated versus a standard curve prepared by adding specific amounts of FITC MPEG in the same medium. Based on the concentration of FITC MPEG on the ghosts (or the concentration of another suitable label) and the number of cells used to prepare the ghosts, the amount of MPEG per cell (membrane modification density) can be calculated for the given experiment.

An additional method of use of the FPEG approach for the measurement of RBC pegylation is achieved through the use of flow cytometry analysis of the pegylated RBC using a FACScan device. The RBC are directly analyzed for fluorescence intensity through a commercial device. The number of PEG molecules attached to the RBC surface is proportional to the percent of active FPEG in the active PEG. The FACScan intensity is connected to the PEG content through a standard curve of pegylation done either through the method above or by comparison to beads containing known amounts of fluorescent molecules on them. Beads used in a FACScan device are commercially available and can be prepared to custom specifications (Bangs Laboratories).

An alternative method for the quantitation of the PEG molecules is the use of radioactively labeled activated MPEG (labeled with covalently attached ³H, ¹⁴C or other appropriate radioactive atom). The red blood cells are washed after the end of the pegylation procedure and then the washed RBC are lysed, decolorized and the radioactivity content is measured through liquid scintillation. The extent of pegylation is calculated using the specific activity of the radiolabeled activated MPEG.

### EXAMPLE 12

### Biological Effects of Pegylation of Bacteria

In order to assess the effects of Pegylation on bacteria, an overnight culture of Yersinia enterocolitica was grown in LB broth. The estimated titer of the overnight culture was approximately 9 log cfu/ml based on a 0.1×O.D. reading of 0.15 to 0.2. The bacterial stock was washed twice with HEPES buffer (150 mM HEPES+50 mM NaCl+75 mM dextrose pH 8.0) by centrifugation at 3800 rpm (approximately 4200× g) for 6 min, and adjusted to the original volume with the HEPES buffer. The bacterial solution was diluted 1:1000 in HEPES buffer to achieve 6 logs cfu/mL of the bacteria. SPA PEG was weighed (330 mg) and completely dissolved in HEPES buffer (2700 mL) by vigorous vortexing. Yersinia enterocolitica (300 μL of the 6 logs cfu/mL suspension) was immediately added to the PEG solution to achieve a final count of 5 logs cfu/mL of the bacteria in the reaction. A control (no PEG) reaction was run in a separate tube by adding Yersinia enterocolitica (300 μL of the 6 logs cfu/mL suspension) to HEPES buffer (2700 mL). The tubes were incubated for 2 hours at RT. The samples in both the tubes were washed twice with Adsol post incubation. Aliquots (0.5 mL) were obtained from each tube (t=0 hr, t=2 hr and post Adsol wash). The aliquots were diluted, plated and then incubated at 37° C. overnight. The plates were read for bacterial growth post incubation. The bacteria treated with SPA PEG remained viable, although the colony formation required longer incubation periods and the colony sizes showed more variability than the untreated bacteria. The colonies of the SPA PEG treated bacteria had basically the same morphology as the untreated bacteria and were essentially the same titer (4.8 for the treated vs. 5.4 for untreated after an Adsol washing of the bacteria).

The bacteria can be assessed for their ability to bind antibody to a particular bacterial antigen (antibodies are commercially available, e.g. Research Diagnostics, Flanders, N.J.). In this way, it can be determined if the bacteria are potentially masked from recognition by an individual's immune system upon transfusion. The antigen binding can be assessed as a function of bacterial growth to assess whether the masked bacteria can subsequently be bound by the antibody after some level of cell divisions. It would be possible to similarly treat the bacteria with a fluorescent or radioactive labeled PEG in order to assess the amount of PEG per bacteria over time of growth of the bacteria to assess what level of PEG modification is required to mask the bacterial antigens and to assess what level of growth is required for a masked bacteria to become unmasked to an individual's immune response. FACScan analysis of the bacteria treated with a fluorescent PEG showed modification of the bacteria with the PEG.

### EXAMPLE 13

The Effect of Buffer Washes on the pH and Extent of Pegylation of Red Blood Cells.

A unit of ABO-typed whole blood (Sacramento Blood Center, Calif.) was leukofiltered according to standard blood banking methods. The red blood cells (RBC) were spun (3800 rpm (4097× g) for 6 min) and the plasma was removed. The cells were then aliquoted into three different tubes.

The effect of washing on the extracellular pH and the amount of pegylation was studied by either washing four times with a bufefr volume 14× the volume of red blood cells, washing two times with 1× the volume of red blood cells or not washing the red blood cells with any buffer.

In the first treatment, the red blood cells (2 mL, approximately 85% HCT) were washed four times with 14× volume of pH=8.0 HEPES buffer (150 mM HEPES, 50 mM NaCl). SPA PEG (221 mg) was weighed and dissolved completely in HEPES buffer (1.3 mL) by vigorous vortexing. The dissolved PEG was immediately added to the washed red blood cells and mixed. A control reaction (no PEG) was also run in a separate container.

In the second treatment, the red blood cells (7.5 mL, approximately 85% HCT) were washed two times with a volume of HEPES buffer equal to the RBC volume. SPA PEG (825 mg) was weighed and dissolved completely in HEPES buffer (7.5 mL) by vigorous vortexing. The dissolved PEG was immediately added to the washed red blood cells and mixed. A control reaction (no PEG) was also run in a separate container. In tube three, unwashed red blood cells (7.5 mL) were used. SPA PEG (825 mg) was weighed and dissolved completely in HEPES buffer (7.5 mL) by vigorous vortexing. The dissolved PEG was immediately added to the red blood cells and mixed. A control reaction (no PEG) was also run in a separate tube.

The reaction was allowed to proceed for 1 hr at RT in each of the three tubes and the cells were subsequently washed with 2× volume of blood bank saline to remove the reaction side products.

Aliquots were removed from each tube after each wash to determine the pH and extent of pegylation. A standard calibrated pH meter was used for these measurements. Control experiments showed the presence of PEG did not affect the pH values measured.

Extracellular pH Values are indicated in the following table:

| | Treatment 1 | Treatment 2 | Treatment 3 |
|---|---|---|---|
| Treatment | Test & Control | Test & Control | Test & Control |
| Wash 0 | 7.18 | 7.18 | 7.18 |
| Wash 1 | 7.99 | 7.76 | — |
| Wash 2 | 8.00 | 7.87 | — |
| Wash 3 | 7.99 | — | — |
| Wash 4 | 7.98 | — | — |
| After Incubation 1 hr | | | |
| Test | 7.70 | 7.66 | 7.46 |
| Control | 7.93 | 7.86 | 7.66 |

The pH of the red blood cells remained constant at approximately pH 8 when washed four times with 14× and two times with 1× buffer. The results indicate that a higher pH is maintained after the incubation with PEG (or without PEG control) in samples that incorporate the wash steps than in the sample with no wash step. It is also observed that the samples with PEG result in a lower pH than the controls, underlying the need for better pH control of the PEG process. The results of pegylation on these samples showed, quantitatively, that pegylation was similar when a high or a low washing volume was used. Not washing the red blood cells however, resulted in substantial decrease in the rate and extent of pegylation.

We claim:

1. A composition of red blood cells comprising:
red blood cells suspected of containing a pathogen wherein the red blood cell composition has been treated such that the pathogen is substantially inactivated and wherein red blood cell antigens are substantially masked so that the transfusion of the treated red blood cells into an antigen mismatched animal would result in a reduced immune reaction compared to the immune reaction of the transfusion of an untreated red blood cell composition, wherein the treated red blood cell composition is suitable for in vivo use.

2. The composition of claim 1, wherein in vivo survival of the red blood cells after circulating for 24 hours following transfusion is greater than 75%.

3. The composition of claim 2, wherein said in vivo survival of greater than 75% is maintained after storage of the red blood cells for up to 14 days at 4° C.

4. The composition of claim 2, wherein said in vivo survival of greater than 75% is maintained after storage of the red blood cells for up to 35 days at 4° C.

5. The composition of claim 2, wherein said in vivo survival of greater than 75% is maintained after storage of the red blood cells for up to 42 days at 4° C.

6. The composition of claim 1, wherein the red blood cell antigens that are substantially masked are minor antigens.

7. The composition of claim 1, wherein when the red blood cells are Rh positive, in vitro binding of anti-D antibody to the treated red blood cells is reduced by at least 90% compared to the untreated red blood cells.

8. The composition of claim 1, wherein when the red blood cells are Rh positive, in vitro binding of anti-D antibody to the treated red blood cells is reduced by at least 95% compared to the untreated red blood cells.

9. The composition of claim 1, wherein when the red blood cells are Rh positive, in vitro binding of anti-D antibody to the treated red blood cells is reduced by at least 99% compared to the untreated red blood cells.

10. The composition of claim 1, wherein when a pathogen is present, at least 3 logs of said pathogen has been inactivated.

11. The composition of claim 10, wherein said pathogen is a bacterium.

12. A composition of red blood cells comprising: red blood cells suspected of containing a pathogen wherein the red blood cell composition has been treated with a compound having an affinity for nucleic acids and an effector group that reacts to bond covalently to the nucleic acid such that the pathogen is substantially inactivated and wherein the red blood cell composition has been reacted with an antigen masking compound such that the red blood cell antigens are substantially masked such that the transfusion of the treated red blood cells into an antigen mismatched animal would result in a reduced immune reaction compared to the immune reaction of the transfusion of an untreated red blood cell composition, wherein the treated red blood cell composition is suitable for in vivo use.

13. The composition of claim 12, wherein said compound having an affinity for nucleic acids comprises a nucleic acid binding ligand.

14. The composition of claim 13, wherein said effector group is selected from the group consisting of a mustard group and a mustard group equivalent.

15. The composition of claim 14, wherein said antigen masking compound comprises polyethylene glycol.

16. The composition of claim 14, wherein said antigen masking compound comprises a polyethylene glycol derivative.

17. The composition of claim 14, wherein said antigen masking compound is selected from the group consisting of an activated polyethylene glycol and an activated polyethylene glycol derivative.

18. The composition of claim 12, wherein said compound having an affinity for nucleic acids is selected from the group consisting of quinacrine mustard and β-alanine, N-(acridin-9-yl), 2-[bis(2-chloroethyl) amino]ethyl ester and wherein the antigen masking compound is selected from the group consisting of 2,2,2-trifluoroethanesulphonyl monomethoxy polyethylene glycol, N-hydroxy succinimide propionic acid monomethoxy polyethylene glycol, and N-hydroxy succinimide butanoic acid monomethoxy polyethylene glycol.

19. The compostion of claim 18, wherein said compound having an affinity for nucleic acids is β-alanine, N-(acridin-9-yl), 2-[bis(2-chloroethyl) amino]ethyl ester.

20. An ex vivo method of treating a red blood cell composition comprising:
(a) contacting the red blood cell composition with a compound that substantially inactivates a pathogen that may be present in the composition, under conditions that result in substantial inactivation of the pathogen present, if any; and
(b) contacting the red blood cell composition with a compound that binds to the red blood cells and substantially masks red blood cell antigens under conditions that significantly reduce the immunogenicity of the red blood cells such that transfusing the red blood cell composition into an antigen mismatched animal would result in a reduced immune reaction compared to the immune reaction of transfusing an untreated red blood cell composition.

21. The method of claim 20, wherein said compound that inactivates a pathogen has an affinity for nucleic acids.

22. The method of claim 21, wherein said compound that inactivates a pathogen comprises an effector group that reacts to bond covalently to the nucleic acid.

23. The method of claim 22, wherein said compound that inactivates a pathogen comprises a nucleic acid binding ligand.

24. The method of claim 23, wherein said effector group is selected from the group consisting of a mustard group and a mustard group equivalent.

25. The method of claim 20, wherein said compound that binds to the red blood cells comprises polyethylene glycol.

26. The method of claim 20, wherein said compound that binds to the red blood cells comprises a polyethylene glycol derivative.

27. The method of claim 20, wherein said compound that binds to the red blood cells is selected from the group consisting of an activated polyethylene glycol and an activated polyethylene glycol derivative.

28. A method of using the composition of claim 1 comprising delivery of the composition into an individual in need of a red blood cell transfusion.

29. A method of using the composition of claim 2 comprising delivery of the composition into an individual in need of a red blood cell transfusion.

30. A method of using the composition of claim 7 comprising delivery of the composition into an individual in need of a red blood cell transfusion

31. A method of using the composition of claim 10 comprising delivery of the composition into an individual in need of a red blood cell transfusion

32. A method of using the composition of claim 11 comprising delivery of the composition into an individual in need of a red blood cell transfusion

33. A method of using the composition of claim 12 comprising delivery of the composition into an individual in need of a red blood cell transfusion

34. A method of using the composition of claim 14 comprising delivery of the composition into an individual in need of a red blood cell transfusion

35. A method of using the composition of claim 17 comprising delivery of the composition into an individual in need of a red blood cell transfusion

36. A method of using the composition of claim 18 comprising delivery of the composition into an individual in need of a red blood cell transfusion

37. A method of using the composition of claim 19 comprising delivery of the composition into an individual in need of a red blood cell transfusion

38. An ex vivo method of treating a red blood cell composition comprising:
(a) providing a red blood cell composition suspected of containing a bacterium, wherein said bacterium, if present, is reacted with an antigen masking compound such that the bacterium is more infectious than a bacterium that is not reacted with the antigen masking compound,
(b) contacting the red blood cell composition with a compound that substantially inactivates the bacterium that may be present in the composition, under conditions that result in substantial inactivation of the bacterium present, if any; and
(c) contacting the red blood cell composition with a sufficient amount of the antigen masking compound such that the antigen masking compound binds to the red blood cells and substantially masks red blood cell antigens under conditions that significantly reduce the immunogenicity of the red blood cells such that transfusing the red blood cell composition into an antigen mismatched animal would result in a reduced immune reaction compared to the immune reaction of transfusing an untreated red blood cell composition.

39. The method of claim 38, wherein said compound that inactivates the bacterium has an affinity for nucleic acids.

40. The method of claim 39, wherein said compound that inactivates the bacterium comprises an effector group that reacts to bond covalently to the nucleic acid.

41. The method of claim 40, wherein said compound that inactivates the bacterium comprises a nucleic acid binding ligand.

42. The method of claim 41, wherein said effector group is selected from the group consisting of a mustard group and a mustard group equivalent.

43. The method of claim 38, wherein said antigen masking compound comprises polyethylene glycol.

44. The method of claim 38, wherein said antigen masking compound comprises a polyethylene glycol derivative.

45. The method of claim 38, wherein said antigen masking compound is selected from the group consisting of an activated polyethylene glycol and an activated polyethylene glycol derivative.

46. A red blood cell processing system comprising:
a) a composition of red blood cells suspected of containing a pathogen wherein the red blood cell composition has been treated such that the pathogen is substantially inactivated and wherein red blood cell antigens are substantially masked so that the transfusion of the treated red blood cells into an antigen mismatched animal would result in a reduced immune reaction compared to the immune reaction of an untreated red blood cell composition, and
b) a blood bag containing the red blood cell composition, wherein the red blood cell composition is suitable for delivery to an individual.

47. The system of claim 46, wherein in vivo survival of the red blood cells after circulating for 24 hours following transfusion is greater than 75%.

48. The system of claim 47, wherein said in vivo survival of greater than 75% is maintained after storage of the red blood cells for up to 14 days at 4° C.

49. The system of claim 47, wherein said in vivo survival of greater than 75% is maintained after storage of the red blood cells for up to 35 days at 4° C.

50. The composition of claim 47, wherein said in vivo survival of greater than 75% is maintained after storage of the red blood cells for up to 42 days at 4° C.

51. The system of claim 46, wherein when the red blood cells are Rh positive, in vitro binding of anti-D antibody to the treated red blood cells is reduced by at least 90% compared to the untreated red blood cells.

52. The system of claim 46, wherein when the red blood cells are Rh positive, in vitro binding of anti-D antibody to the treated red blood cells is reduced by at least 95% compared to the untreated red blood cells.

53. The system of 46, wherein when the red blood cells are Rh positive, in vitro binding of anti-D antibody to the treated red blood cells is reduced by at least 99% compared to the untreated red blood cells.

54. The system of claim 46, wherein when a pathogen is present, at least 3 logs of said pathogen has been inactivated.

55. The system of claim 54, wherein said pathogen is a bacterium.
